(19) United States
(12) Patent Application Publication
(10) Pub. No.: US 2026/0315247 A1
(43) Pub. Date: Oct. 15, 2026

(54) SPATIOTEMPORALLY PATTERNED MULTI-CHANNEL MICROSTIMULATION OF SOMATOSENSORY CORTEX FOR IMPROVED RESOLUTION FORCE FEEDBACK AND PERCEPTION OF TACTILE MOTION AND EDGE ORIENTATION

(71) Applicants: THE UNIVERSITY OF CHICAGO, Chicago, IL (US); UNIVERSITY OF PITTSBURGH - OF THE COMMONWEALTH SYSTEM OF HIGHER EDUCTION, Pittsburgh, PA (US)

(72) Inventors: Sliman BENSMAIA, Chicago, IL (US); Charles Greenspon, Chicago, IL (US); Giacomo Valle, Chicago, IL (US); Rob Gaunt, Pittsburgh, PA (US); Taylor Hobbs, Pittsburgh, PA (US); Cecile Verbaarschot, Pittsburgh, PA (US); Robin Lienkamper, Pittsburgh, PA (US)

(21) Appl. No.: 19/485,812

(22) PCT Filed: Jun. 14, 2024
(86) PCT No.: PCT/US2024/034216

§ 371 Date: Nov. 19, 2025

Related U.S. Application Data
(60) Provisional application No. 63/508,761, filed on Jun. 16, 2023.
(60) Provisional application No. 63/558,449, filed on Feb. 27, 2024.

(51) Int. Cl. A61F 2/54; A61N 1/05; A61N 1/36

(52) U.S. Cl. CPC A61F 2/54; A61N 1/0531; A61N 1/36003; A61N 1/36139

(57) ABSTRACT

Embodiments are provided for stimulation of somatosensory cortex such that (i) the perceptions of force induced thereby exhibit increased levels of discrimination, allowed for finer-resolution perceptions to be achieved, (ii) perceptions of edges or other geometric aspects of objects can be delivered, and (iii) perceptions of motion of objects can be delivered. These embodiments include providing such stimulation to multiple electrodes or other stimulation sites synchronously, with the multiple stimulation sites evoking perceptions whose locations on the hand at least partially overlap. Perception of edges can be evoked by synchronously stimulating multiple stimulation sites somatotopically oriented along the orientation of the edge. Perception of motion can be evoked by providing sequential stimulus from one stimulation site to another, with the interval between sequential stimuli non-overlapping enough to induce perception of motion while close enough in time to evoke a single percept rather than a sequence of discrete percepts.

### CROSS-REFERENCE TO RELATED APPLICATION

This application claims priority to U.S. provisional application No. 63/508,761, filed Jun. 16, 2023, and U.S. provisional application No. 63/558,449, filed Feb. 27, 2023, the contents of which are hereby incorporated by reference.

### STATEMENT REGARDING FEDERALLY SPONSORED RESEARCH OR DEVELOPMENT

This invention was made with government support under NS122333, and NS107714 awarded by the National Institutes of Health. The government has certain rights in the invention.

### BACKGROUND

A variety of injuries can result in reduction or loss of motor control and/or sensory input, e.g., spinal cord injuries. To restore lost motor and/or sensory function, the residual nervous system can be accessed and stimulated/recorded at a variety of locations along the neuroaxis (e.g., peripheral nerves, cortex, and spinal cord). The activity of the nervous system could be recorded in order to provide control outputs (e.g., for a robotic assistive device, for a prosthetic limb, to electrically stimulate a paralyzed limb). The nervous system could be stimulated to provide sensory feedback (e.g., a sensation of force, temperature) related to parameters experienced by a real or artificial limb and/or information related to an assistive system. In practice, it is difficult to implement a sensory prosthetic system that provides clinically relevant and natural perceptions while remaining with safe constraints with respect to stimulation limits (e.g., magnitude of current injected, frequency of stimulation).

### SUMMARY

In a first aspect, a system is provided that includes: (i) an implantable multichannel array that comprises a plurality of channels, each configured to provide stimulation to a respective portion of a somatosensory cortex of a user in which the multichannel array is implanted; and (ii) a controller that is operably coupled to the implantable multichannel array and that comprises one or more processors.

The controller is configured to perform controller operations. The controller operations can comprise: (i) obtaining a first time-varying input force; (ii) based on the first time-varying input force, determining a first time-varying stimulus profile; and (iii) providing, via a first subset of channels of the multichannel array, synchronized stimulation having a magnitude that varies according to the first time-varying stimulus profile, wherein the first subset of channels consists of channels that provide stimulation to respective portions of the somatosensory cortex of the user such that respective perceived locations of the stimulus provided by each channel of the first subset of channels at least partially overlap. The controller operations can additionally or alternatively comprise:

The controller operations can additionally or alternatively comprise: (i) obtaining at least one of a direction or a speed of a motion; (ii) based on the direction or speed of the motion, determining a first time-varying stimulus profile for a first channel of the multichannel array and a second time-varying stimulus profile for a second channel of the multichannel array, wherein the first time-varying stimulus profile includes an amount of stimulus prior to stimulus of the second time-varying stimulus profile, and wherein identities of the first channel and second channel are selected based on the direction of the motion; (iii) providing, via the first channel, stimulation having a magnitude that varies according to the first time-varying stimulus profile; and (iv) providing, via the second channel, stimulation having a magnitude that varies according to the second time-varying stimulus profile.

The controller operations can additionally or alternatively comprise: (i) obtaining at least one of a direction or a speed of a motion; (ii) based on the direction or speed of the motion, determining a first time-varying stimulus profile for a first channel of the multichannel array and a second time-varying stimulus profile for a second channel of the multichannel array, wherein the first time-varying stimulus profile includes an amount of stimulus prior to stimulus of the second time-varying stimulus profile, and wherein identities of the first channel and second channel are selected based on the direction of the motion; (iii) providing, via the first channel, stimulation having a magnitude that varies according to the first time-varying stimulus profile; and (iv) providing, via the second channel, stimulation having a magnitude that varies according to the second time-varying stimulus profile.

In a second aspect, a method is provided that includes: (i) obtaining a first time-varying input force; (ii) based on the first time-varying input force, determining a first time-varying stimulus profile; and (iii) providing, via a first subset of channels of an implantable multichannel array implanted in a somatosensory cortex of a user, synchronized stimulation having a magnitude that varies according to the first time-varying stimulus profile, wherein the first subset of channels consists of channels that provide stimulation to respective portions of the somatosensory cortex of the user such that respective perceived locations of the stimulus provided by each channel of the first subset of channels at least partially overlap.

In a third aspect, a method is provided that includes: (i) obtaining at least one of a direction or a speed of a motion; (ii) based on the direction or speed of the motion, determining a first time-varying stimulus profile for a first channel of an implantable multichannel array and a second time-varying stimulus profile for a second channel of the multichannel array, wherein the first time-varying stimulus profile includes an amount of stimulus prior to stimulus of the second time-varying stimulus profile, and wherein identities of the first channel and second channel are selected based on the direction of the motion; (iii) providing, via the first channel, stimulation having a magnitude that varies according to the first time-varying stimulus profile; and (iv) providing, via the second channel, stimulation having a magnitude that varies according to the second time-varying stimulus profile.

In a fourth aspect, a method is provided that includes: (i) obtaining an orientation of an edge; (ii) based on the edge, determining a first stimulus profile; and (iii) providing, via a first subset of channels of an implantable multichannel array, synchronized stimulation having a magnitude that corresponds to the first stimulus profile, wherein the first subset of channels consists of channels that provide stimulation to respective portions of a somatosensory cortex of a user such that respective perceived locations of the stimulus provided by each channel of the first subset of channels are arranged along a direction based on the orientation of the edge.

In a fifth aspect, a non-transitory computer readable medium is provided having stored thereon program instructions executable by at least one processor to cause the at least one processor to perform the method of the second, third, or fourth aspects.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings are included to provide a further understanding of the system and methods of the disclosure and are incorporated in and constitute a part of this specification. The drawings illustrate one or more embodiment(s) of the disclosure, and together with the description serve to explain the principles and operation of the disclosure.

FIG. 1A depicts aspects of an example system.

FIG. 1B depicts aspects of operation of the example system of FIG. 1A, according to example embodiments.

FIG. 1C depicts aspects of operation of the example system of FIG. 1A, according to example embodiments.

FIG. 1D depicts aspects of operation of the example system of FIG. 1A, according to example embodiments.

FIG. 1E depicts aspects of operation of the example system of FIG. 1A, according to example embodiments.

FIG. 1F depicts aspects of operation of the example system of FIG. 1A, according to example embodiments.

FIG. 1G depicts aspects of operation of the example system of FIG. 1A, according to example embodiments.

FIG. 1H depicts aspects of operation of the example system of FIG. 1A, according to example embodiments.

FIG. 1I depicts aspects of operation of the example system of FIG. 1A, according to example embodiments.

FIG. 1J depicts aspects of operation of the example system of FIG. 1A, according to example embodiments.

FIG. 1K depicts aspects of operation of the example system of FIG. 1A, according to example embodiments.

FIG. 2 depicts aspects of an example system.

FIG. 3A depicts aspects of an example method.

FIG. 3B depicts aspects of an example method.

FIG. 3C depicts aspects of an example method.

FIG. 4 depicts experimental results.

FIG. 5 depicts experimental results.

FIG. 6 depicts experimental results.

FIG. 7 depicts experimental results.

FIG. 8 depicts experimental results.

FIG. 9 depicts experimental results.

FIG. 10 depicts experimental results.

FIG. 11 depicts experimental results.

FIG. 12 depicts experimental results.

FIG. 13 depicts experimental results.

FIG. 14 depicts experimental results.

FIG. 15 depicts experimental results.

FIG. 16A depicts experimental results.

FIG. 16B depicts experimental results.

FIG. 17A depicts experimental results.

FIG. 17B depicts experimental results.

FIG. 17C depicts experimental results.

FIG. 18A depicts experimental results.

FIG. 18B depicts experimental results.

FIG. 18C depicts experimental results.

FIG. 18D depicts experimental results.

FIG. 19A depicts experimental results.

FIG. 19B depicts experimental results.

FIG. 19C depicts experimental results.

FIG. 20A depicts experimental results.

FIG. 20B depicts experimental results.

FIG. 20C depicts experimental results.

FIG. 21A depicts experimental results.

FIG. 21B depicts experimental results.

FIG. 22A depicts experimental results.

FIG. 22B depicts experimental results.

FIG. 22C depicts experimental results.

FIG. 23A depicts experimental results.

FIG. 23B depicts experimental results.

### DETAILED DESCRIPTION

The following detailed description describes various features and functions of the disclosed systems and methods with reference to the accompanying figures. The illustrative system and method embodiments described herein are not meant to be limiting. It may be readily understood that certain aspects of the disclosed systems and methods can be arranged and combined in a wide variety of different configurations, all of which are contemplated herein.

### I. Overview

It is desirable in a variety of scenarios to artificially provide sensory feedback to a person, e.g., to provide the sensation of a force and/or properties (e.g., motion, geometry, texture) of an object in contact with the person's body (or a prosthetic device, e.g., a prosthetic hand) at a particular location of the person's body. Such a sensation could be related to a force, shape, vibration motion, or other tactile interaction exerted onto a prosthetic device (e.g., a prosthetic hand provided to a person who has lost part of an upper limb), the user's own body (e.g., a subdermal force or other tactile sensor implanted in the user's finger, or a force or other tactile sensor worn on the user's finger, to provide and/or restore sensation in the face of a spinal cord injury), an assistive device (e.g., a robotic hand attached to a wheelchair), or a force, edge, motion, vibration, or other tactile sensation that is based on some other input (e.g., sensory feedback from a communications device). A variety of different stimulation locations (peripheral nerves, thalamus, and cortex) and stimulation methods (electrical, optical, various waveforms and mappings from force to waveform properties) have been attempted. However, it can be difficult to evoke useful and naturalistic levels of force sensation or other tactile percepts (e.g., motion of an object along the skin, an edge or other geometry of an object in contact with the skin) while remaining within safe stimulation limits (e.g., with respect to total injected charge).

Note that variously throughout, reference may be made to detecting, or providing stimulus to evoke a perception of, forces on a body part and/or properties of an object in contact with a body part (e.g., the motion of such an object along the body part, edges, concavity/convexity, or other aspects of the geometry of such an object in contact with the body part). It is to be understood that such mention throughout this disclosure is intended to represent detecting and/or evoking the perception of such phenomena both in the context of a real human body part, in the context of an artificial body part (e.g., a finger or other element of a prosthetic hand, or some other element of some other prosthetic system), in the context of a simulated body part (e.g., a body part in a virtual environment), and/or in the context of evoking such perceptions in an manner abstract from any specific real or virtual body part or other object (e.g., providing a pattern or forces, edges, motions, or other perceptions as part of a user interface or other application for providing information to a person via stimulation of the somatosensory cortex).

The systems and methods described herein provide for improved stimulation of the somatosensory cortex (e.g., the primary somatosensory cortex), leading to the ability to provide a variety of different percepts and/or improvements to such percepts. This includes the ability to induce the perception of edges (including controlling the orientation, location, and other aspects of the geometry of the perceived edges), the ability to perceive motion along a body part (e.g., of an object in contact with the body part moving along the surface of the body part), the ability to provide perceptions of force with high degrees of discriminability (e.g., more than 20 perceivably different levels of the force stimulus), the ability to perceive the concavity or convexity of an object in contact with a body part, the ability to perceive various shapes or icons as a pattern of motion along a body part, or other percepts.

For example, the systems and methods described herein provide for the evocation of sensory perceptions that exhibit improved discriminability. This allows higher-resolution force percepts to be delivered to a person, allowing them to receive more fine-grained sensation of force, e.g., as detected using a force sensor in the finger of a prosthetic hand or some other source of force input. These improvements are provided, in part, by providing synchronized stimulation, via multiple channels (e.g., electrodes, light-emitting stimulators) of a multichannel array, to multiple portions of somatosensory cortex that represent different, but at least partially overlapping, locations of perceived stimulus when stimulated individually.

This results in a perceived stimulus at a location corresponding to the overlapping perceptive locations of each of the individual stimulation channel sites (e.g., a location that is overlapped by all of the perceptive locations, or an average location nearby) that can exhibit greater intensity and higher resolution (with respect to, e.g., the number of levels of just noticeable difference (JND) that a person can perceive as the magnitude of the stimulation is varied). These improvements may be related to the fact that, by providing synchronized stimulation through multiple channels, the total amount of provided stimulus can be increased while remaining within specified safety limits (e.g., maximum charge injection limits) with respect to the stimulation provided by any individual channel. Additional benefits with respect to the intensity and resolution of the perceived stimulus can be provided by translating the input force signal into a stimulus profile in a manner that emphasizes the effect of transients or other changes in the input signal (e.g., sudden positive or negative edges in the input signal) on the magnitude of the applied synchronized stimulation.

The systems and methods described herein can also be used to evoke tactile sensory perceptions of edges (e.g., of edges of objects in contact with a body part and/or a part of a prosthetic device). The perception of an edge can be induced by providing synchronized stimulation across a set of stimulation sites whose corresponding projected fields are located at and oriented along the location and orientation of the edge. For example, if an edge (e.g., an edge of an object in contact with a fingertip of a prosthetic hand) is detected, synchronized stimulation could be provided to a corresponding set of stimulation sites whose projected fields (e.g., with respect to a mapping thereof from a user's natural hang to the prosthetic hand) are located along a line located at the location of the detected edge and oriented along the orientation of the detected edge.

The systems and methods described herein can also be used to evoke tactile sensory perceptions of motion (e.g., of motion of an object in contact with a body part along a surface of the body part). This can be accomplished by sequentially providing stimulation via a series of stimulation sites (e.g., along a trajectory of projected fields whole locations correspond to the location and orientation of the motion of the object). To provide a sensation of motion, and not of a constant contact that is non-perceptibly moving and/or of a series of discrete contacts, the timing of the stimulation through adjacent-in-time stimulation sites could be specified such that an inter-stimulus interval is less than a maximal latency (e.g., less than 500 milliseconds, or less than 400 milliseconds) and further such that the stimulus from adjacent-in-time stimulation sites does not overlap by more than a threshold duration of time (e.g., by less than 25 milliseconds, or such that no more than one or two final pulses of stimulation from an initial site overlap in time with onset of stimulation from a subsequent site).

The systems and methods described herein can also be used to evoke additional or alternative tactile sensory perceptions related to the geometry, texture, or other tactile features of an object or other tactile percepts. For example, a degree of concavity or convexity of an object in contact with a body part could be evoked by providing, through three or more stimulation sites, a spatiotemporal pattern of stimulation to simulate contact (and optionally, termination of contact) with such an object. For example, such that ‘middle’ site(s) start providing stimulation earlier and provide higher-magnitude stimulation that ‘outer’ sites (simulating the fact that convex objects will contact the middle of a contact area first, and exert a higher force on the hand while in contact). In another example, geometric patterns of motion could be provided in order to indicate letters or other symbols in a manner that is more accurately distinguishable than, e.g., providing simultaneous stimulation though the same set of stimulation sites.

Stimulation can be provided according to the various modes described herein (e.g., increased-discrimination force, motion, edge, geometry) at the same time. For example, if a force exerted by an object onto a prosthetic hand is detected along with an edge of that object and also a degree or motion of slip of the object along the prosthetic hand, spatiotemporal patterns of stimulation could be determined as described herein for the evocation of the perception of motion, edge, and high-discriminability force and these various patterns could be combined (e.g., as a linear combination of the patterns, as a nonlinear weighted sum of the patterns) in order to deliver simultaneously evoke all of the percepts. Alternatively, a sensory stimulation system could operation by switching between modes according to an application in order to provide such percepts during different, non-overlapping periods of time. For example, during an initial contact between an object and a prosthetic hand, one or more force or other tactile sensors could be operated to detect that an edge or other geometry is present on the part of the object that contacted the hand. As a result, during a first period of time, a spatiotemporal pattern of stimulation could be provided to evoke a perception of the edge, with an orientation corresponding to the detected orientation of the edge as it contacts the hand. Subsequently, to facilitate fine control or manipulation of the object, a spatiotemporal pattern of stimulation could be provided during a second period of time such that synchronized stimulation is provided to a set of stimulation sites whose projected fields overlap and correspond to a location of contact with the object in order to provide a perception of force that exhibits an enhanced degree of discriminability with respect to the magnitude of the perceived force. Then, in response to a sensor (e.g., a vibration sensor, an optical flow sensor) detecting motion of the object relative to the hand (e.g., due to slippage of the object), a spatiotemporal pattern of stimulation could be provided during a third period of time to provide a perception of motion whose speed and orientation correspond to the detected speed and orientation of the motion of the object.

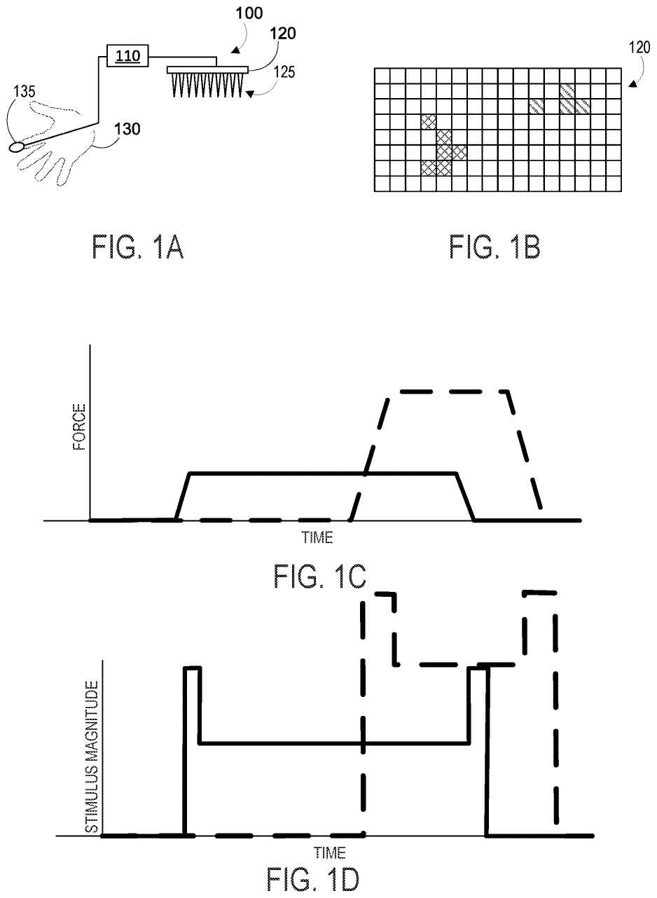

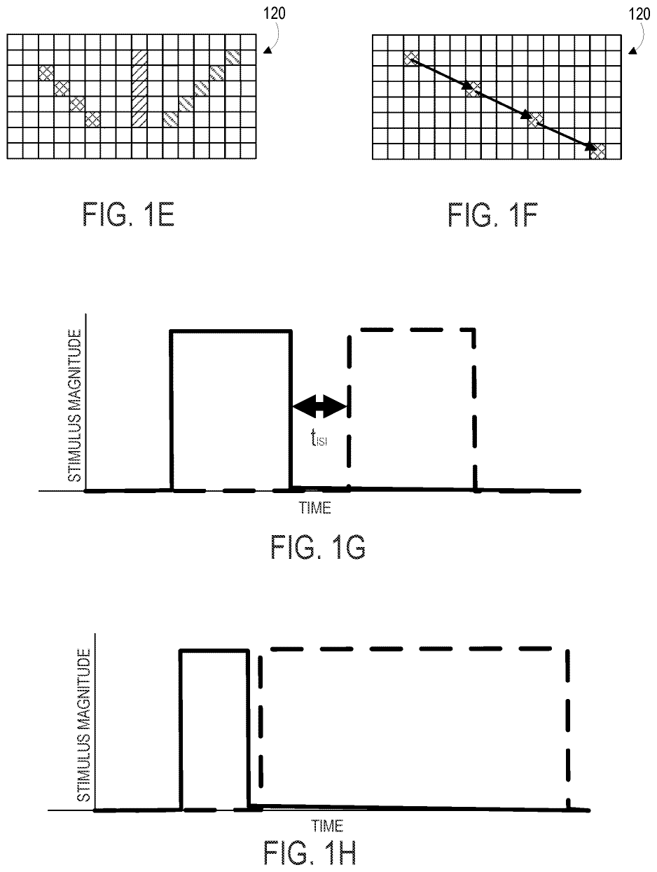

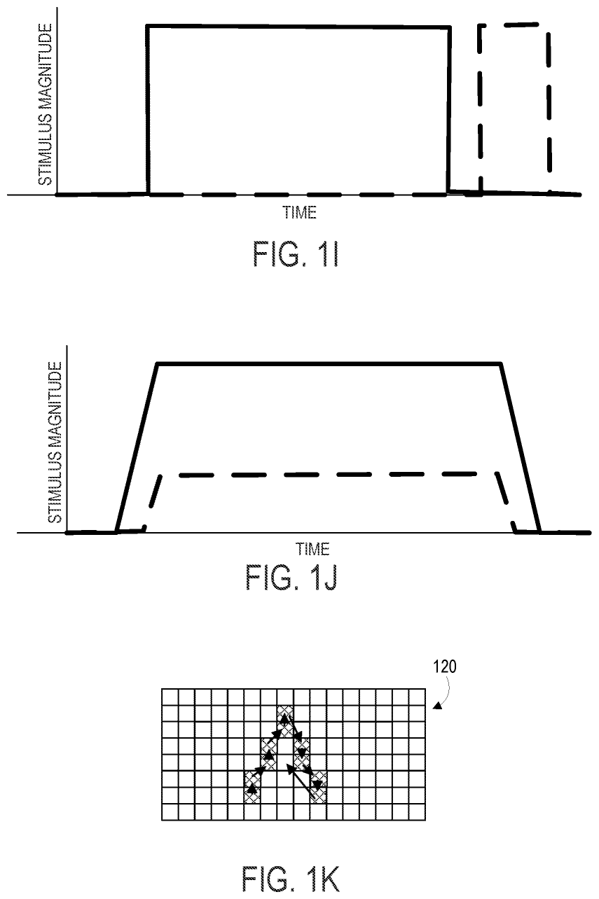

FIG. 1A illustrates, by way of a non-limiting example embodiment, elements of a system 100 that can be used to detect an input force signal or other input tactile information (edges, motion, geometry, etc.) and to provide, via electrical or other stimulation to the somatosensory cortex (e.g., a primary somatosensory cortex) of a user, sensations related to that input force signal or other tactile information. The system includes a controller 110, an implantable multichannel array 120 that has been implanted into the somatosensory cortex of the user, and a sensor 135 that is operable to detect a force.

The controller 110 may be a single controller embodied in a single device, or multiple controllers which may be embodied in one or more devices. For example, the controller 110 could include a first controller disposed within a prosthetic hand 130 that is configured, among other operations (e.g., to operate actuators of the hand 130 to perform actions like gripping, pressing buttons, etc.), to operate the sensor 135 to generate a time-varying input force signal or other tactile information therefrom. The controller 110 could also include a second controller disposed within an implantable device/system that includes the multichannel array 120 and that is configured, among other operations, to receive an indication of the time-varying input force signal or tactile information (or some other signal derived therefrom, e.g., a time-varying stimulus profile or other spatiotemporally patterned stimulation) and to operate the multichannel array 120 to provide stimulus (e.g., by injecting time-varying currents through electrodes or other types of channels of the multichannel array 120) sufficient to result in the user perceiving a time-varying force or other tactile percept related to the input time-varying input force signal and/or other input tactile information. The first and second controllers could be in direct communication (e.g., via a wireless communications link) or could be in communication via one or more additional controllers or other systems of the controller 110. For example, the first and second controllers could be in communication with a master controller unit that manages communication between the various elements of the system 100, e.g., between one or more prosthetics like the hand 130, one or more implanted stimulator and/or detector units, and/or a wheelchair or other assistive device(s).

The implantable multichannel array 120 includes a plurality of individual channels that are operable to provide stimulation (e.g., electrical stimulation, optical stimulation) to respective portions of a somatosensory cortex into/on which the implantable multichannel array 120 has been implanted. As illustrated in FIG. 1A, the implantable multichannel array 120 could include a plurality of penetrating stimulation channels 125 which could include, e.g., respective individual electrode sites, light emitters (e.g., LEDs, ends of optical fibers or other optical waveguides that transmit light to the end of the penetrating element from a light emitter at the base), or other stimulus-providing elements at or near the ends of the respective penetrating elements. Additionally or alternatively, the implantable multichannel array 120 could include non-penetrating stimulation channels (e.g., surface electrodes for providing electrical stimulus at the surface of the cortex, lasers or other light emitters for providing optical stimulus to the cortex from the surface of the cortex).

The implantable multichannel array 120 providing stimulus though a single channel of the array can result in the user perceiving a force or other tactile percept at a particular location, e.g., an area of skin of the user's body. Such a location could be a location of a portion of the user's body that currently is part of the user's body (e.g., a finger of a paralyzed arm) or a portion of the user's body that has been lost (e.g., a finger of a limb that has been amputated or otherwise lost). The location where the user perceives stimulus provided via a particular channel can be related to the portion of the somatotopic map of the user's somatosensory cortex that is stimulated by the particular channel.

The size of the implantable multichannel array 120, the spacing of its channels, the range of the user's somatotopic map stimulated by any particular channel, and the spatial resolution of the user's somatotopic map (e.g., the area spanned by the portion of the user's somatotopic map to represent the user's hands) can result in the locations of the perceived stimulation evoked by each channel partially or fully overlapping. For example, stimulation provided by a first channel could result in a perceived stimulus having a location that spans the entire ventral surface of the end segment of the user's index finger, while stimulation provided by a second channel could result in a perceived stimulus having a location that spans the proximal half of the ventral surface of the end segment of the user's index finger and the terminal half of the ventral surface of the adjacent segment of the user's index finger; both of the stimulus locations overlap with respect to the proximal half of the ventral surface of the end segment of the user's index finger. Where sets of two or more (e.g., of four or more) channels of the multichannel array 120 overlap with respect to the locations of their respective evoked perceptions, synchronized stimulation can be provided via all of the channels in the set in order to provide improved (e.g., higher intensity and/or higher-resolution) perceptions in time with an input force signal.

During the calibration of such a system (e.g., system 100), the identity of sets of channels of such a multichannel array that overlap in this manner could be determined and/or designated in a clinical setting. The system could then be programmed or otherwise set to provide improved synchronized stimulation to the channels of each set in time with respective input force signal(s). Such a calibration process could include determining, for each channel based on user feedback, the extent of the location on the user's body of the sensation evoked by stimulus provided by each channel. Sets of channels that at least partially overlap could then be determined by identifying sets of channels whose individual sensation locations overlap. This could include determining sets of channels whose evoked sensation locations all overlap the same area on the user's body and/or determining sets of channels whose evoked sensation locations all overlap with the location of at least one other channel in the set (e.g., one channel in such a set could have a location of evoked sensation that overlaps with the location of only one other channel in the set). Additionally or alternatively, the determination that the sensation evoked by pairs (or larger sets) of channels overlap could be determined in a pairwise (or set-wise) fashion, providing stimulation through each channel of a pair (or set) and asking the user if the locations of the evoked perceptions overlap.

FIG. 1B is a schematic map of channels of the multichannel array 120. Note that the number, size, and arrangement of the channels in such an array is intended as a non-limiting example embodiment; for example the channels in such an array could have a hexagonal, circular, or other pattern. Providing stimulus through each of the channels of the array can evoke a corresponding sensation, spanning the area of a corresponding location, of a user's body (note, as above, that such a location may be on a body part that the user has lost, e.g., via amputation). These locations may be mapped to determine sets of the channels whose sensation locations at least partially overlap; additionally or alternatively, such patterns of overlap may be detected directly (e.g., by asking, in a pairwise fashion, whether the locations of sensation evoked by stimulation via a pair of channels overlap). FIG. 1B illustrates the identity of two such sets of channels; the identity of channels of the first set, which includes five channels, is indicated by cross-hatching while the identity of channels of the second set, which includes four channels, is indicated by parallel diagonal lines.

Accordingly, sensations relating to two different input signals (e.g., force signals generated from two different force sensors) may be provided to a user in an improved manner (e.g., exhibiting greater intensity and/or resolution while remaining within a specified maximum stimulation magnitude through any single channel) by providing synchronized stimulation through the sets of channels. This could be accomplished by programming a memory of a system (e.g., an implantable system) with the identity of the channels in each identified set of channels. Such a memory could be accessed by a microprocessor or other controller in order to determine which channels, or a multichannel array, to operate to provide synchronized stimulation in time with a time-varying stimulus profile determined therefor (based, e.g., on a filtered version of a corresponding time-varying input force). This could include the microprocessor operating individual current sources or other stimulus-generating elements for each of the channels in the set and/or operating switches or other elements to connect two or more channels of the set to a common current source or other stimulus-generating element.

Additionally or alternatively, the memory could take the form of programmable switches (which may be reprogrammable, or which may be programmable only once) that determine whether each individual channel is part of a set (or of a particular set of a plurality of sets) of channels and thus should operate to provide synchronized stimulation with the remainder of the set. For example, each channel could be connected to one (or more) current sources or other stimulus-generating elements via a respective one (or more) switch, such that ‘programming’ the switches to reflect to identity of channels in an identified set of channels includes setting the switches of the channels in the set to electrically couple the channels in the set to the current source, while setting the switches of other channels that are not in the set to be disconnected. Each channel could include multiple such switches to allow for the multichannel array to be programmed with multiple such sets of channels (e.g., to provide improved force sensations related to multiple fingertips or other multiple locations of a user's body). Additionally or alternatively, such multiple sets could be implemented by repeatedly reprogramming the switches to allow for synchronized stimulation to be provided via multiple different identified sets of channels during different periods of time.

Providing synchronized stimulation to two or more channels of a multichannel array could include applying the same electrical, optical, or other waveform to all of the channels in common. For example, the channels could be electrodes and providing synchronized stimulation to the two or more channels of a set of channels could include electrically coupling all of the two or more channels to a single common current source or other stimulus waveform signal generator (e.g., via a set of switches that have been set to electrically couple the channels of the set, and no other channels, to the signal generator). However, synchronized stimulation can be provided via two or more channels in other ways, e.g., to account for limitations of the multichannel array, to reduce power requirements, to provide for redundancy, or to provide some other benefit or account for some other factor. For example, the timing of individual pulses of excitatory stimulus emitted by different channels of the set of two or more channels could vary slightly due to imperfections in timing generators used separately by each channel, due to transmission delays along electrical or other signal propagation traces between the channels, due to time-domain multiplexing of transmission lines, channel circuitry, or other components between the channels, or due to some other factors.

For stimulus provided through two or more channels to be “synchronized,” within the meaning of this disclosure, individual pulses of excitatory stimulation (e.g., pulses of current injected into tissue, pulses of light emitted into tissue) delivered from the various channels in a set of two or more channels must have pulse widths that differ from an average of the delivered pulse widths by less than 50% of the average delivered pulse width and pulse timings that differ from an average delivered pulse timing by less than 25% of an average inter-pulse interval of the applied pulses (e.g., less than 25% of a reciprocal of a frequency at which the pulses are delivered, in examples wherein the pulses are delivered periodically at the frequency).

Another way to increase the perceived intensity and/or resolution of sensations delivered by stimulating the somatosensory cortex is to provide stimuli whose magnitude varies with changes in the input force signal in a manner that emphasizes transients or other changes in the input force signal. Since natural biosignals (e.g., cell firing rates) detected in relation to natural changes in experienced force exhibit similar transient-sensitivity, such a scheme for translating time-varying input force signals into time-varying stimulus profiles may be referred to as ‘biomimetic.’ By way of non-limiting illustrative example, FIG. 1C depicts two time-varying input force signals (e.g., force signals detected from respective force sensors at the end of respective fingers of a prosthetic hand). Such signals could be used directly to modulate the magnitude (e.g., current amplitude) of stimulus provided to respective sets of channels of a multichannel array (e.g., to the first and second sets of channels illustrated in FIG. 1B) in order to evoke respective time-varying sensations of force at respective perceived locations on a user's body. Alternatively, the time-varying input force signals could be used to determine respective time-varying stimulus profiles that are increased by increases in the magnitudes of their respective time-varying input force signals and by transient changes in their respective time-varying input force signals. The time-varying stimulus profiles could then be used to provide stimulus to the user's somatosensory cortex via respective sets of two or more channels of an implanted multichannel array (e.g., by setting the current amplitude or other magnitude factor of a repetitive train of stimulus pulses to follow the time-varying stimulus profile).

FIG. 1D depicts, by way of non-limiting illustrative example, two such time-varying stimulus profiles determined from respective time-varying input force signals of FIG. 1C. Such signals could be determined via a weighted combination of the underlying time-varying input force signal and one or more signals derived from the time-varying input force signal in a manner that emphasized transients or other changes thereof. For example, the time-varying stimulus profile could be determined as a weighted combination of the time-varying input force signal and the first derivative (or some other derivative) of the time-varying input force signal. Other transient-sensitive derived signals could be used additionally or alternatively. E.g., an edge detector function, a high-pass filter, a wavelet filter, a nonlinear function, or some other function that exhibits increases in response to changes (e.g., supra-threshold changes) in the time-varying input force signal.

The systems and methods described herein can be used to improve intensity and/or perceptual resolution (e.g., as measured by levels of JND) of force sensations evoked in a user. For example, synchronously driving sets of four or more channels (that overlap with respect to their locations of induced sensation as described above) using time-varying stimulus profiles that are sensitive to transient changes in an input time-varying force signal can result in a user in which the multichannel array is implanted perceiving stimulus in a manner that permits the user to distinguish at least 20 different levels of the stimulus. These effects are further evidenced by the experimental data related below.

Synchronized stimulation provided via a set of channels of such an array can also be used to evoke other perceptions. For example, synchronized stimulation can be provided via all of the channels of a subset of channels of a multichannel array in order to evoke the perception of an edge of a specified orientation. This can be accomplished by selecting the subset of channels such that the channels' projected fields extend across a (real or perceived) surface of a body part along a direction that corresponds to the specified orientation (and optionally whose overall location corresponds to a specified location of the edge, e.g., relative to a prosthetic hand or other body part). The perceptual fields of a selected set of channels could be overlapping or non-overlapping.

FIG. 1E is a schematic map of channels of the multichannel array 120. FIG. 1E illustrates the identity of three subsets of channels; the identity of channels of the first set, which includes five channels, is indicated by cross-hatching, the identity of channels of the second set, which includes four channels, is indicated by lower-left to upper-right parallel diagonal lines, and the identity of channels of the third set, which includes three channels, is indicated by upper-left to lower-right parallel diagonal lines. Providing synchronized stimulus to the channels of the second set could evoke a perception of a ‘vertically’ oriented edge. Providing synchronized stimulus to the channels of the first set could evoke a perception of an edge oriented approximately 45 degrees counter-clockwise from vertical, and providing synchronized stimulus to the channels of the third set could evoke a perception of an edge oriented approximately 45 degrees clockwise from vertical.

The identity of the channels in a particular subset could be determined, based on a commanded edge orientation (and optionally location), based on a lookup or other computation of the locations and geometry of the projected fields of the channels of the multichannel array 120. For example, a grid search, gradient descent, greedy search, or other method could be used to determine a subset of the channels whose perceptual fields span a perceptual space along a commanded orientation (and optionally, do so proximate to a commanded location). Such computations could be performed anew for each commanded orientation (and location), or could be pre-computed (e.g., during a calibration process that also includes determining the perceptual fields for each of the channels) and stored (e.g., in a memory of a prosthetic system) for later use. Once there is a need to evoke the perception of an edge at a specified orientation, the stored patterns could be searched for a pattern that correspond to, or that is sufficiently similar to, the specified orientation.

Evoking the perception of an edge in this manner could be performed as a result of detecting that a user's natural hand, a prosthetic hand, or some other body part has come into contact with an object in a manner that presents an edge (or similar geometric feature) of the object at a particular orientation in contact the body part (e.g., by detecting the edge in a field of forces detected by an array of force sensors on the body part). Additionally or alternatively, the perception of an edge could be evoked to provide sensation related to a simulated object (e.g., in a virtual environment, that has come into contact with a user's virtual hand), to provide an element of a user interface (e.g., to provide an indication of an angle of a lever, slider, or other real or virtual control surface or indicator), or to present abstract information to a user.

Specified spatiotemporal patterns of stimulation provided via channels of such an array can also be used to evoke the perception of motion (e.g., of motion of an object in contact with a body part across a surface of the body part). This can be accomplished by providing stimulation via a number of different channels sequentially whose projected fields are located along a (real or perceived) surface of a body part along a direction of the motion (and optionally whose overall location corresponds to a specified location of the object that is moving, e.g., relative to a prosthetic hand or other body part). The perceptual fields of stimulated channels could be overlapping or non-overlapping.

FIG. 1F is a schematic map of channels of the multichannel array 120. FIG. 1E illustrates the identity of four channels (indicated by cross-hatching) via which stimulation could be provided in sequence (according to the direction of the arrows) in order to evoke a perception of motion in a direction along the perceptual fields of the channels. The identity of the channels could be determined, based on a commanded direction (and optionally location) of motion, based on a lookup or other computation of the locations and geometry of the projected fields of the channels of the multichannel array 120. For example, a grid search, gradient descent, greedy search, or other method could be used to determine a set of channels whose perceptual fields span a perceptual space along a commanded direction of motion (and optionally, do so proximate to a commanded location of the motion).

To ensure that the sequential stimulation provided by such a set of two or more channels is perceived as a motion, and not as, e.g., an imperceptibly moving continuous stimulus or a sequence of discrete taps or other discrete tactile events, the relative timing between the stimulation provided by adjacent sites in the sequence could satisfy one or more constraints. For example, if the stimulation provided by two adjacent sites in the sequence are separated in time by too great a degree, the percept evoked thereby will be perceived as separate taps or some other discrete tactile percepts rather than as a continuous motion. FIG. 1G depicts two stimuli (solid line and dashed line) that could be delivered via respective first and second stimulation sites in a sequence. The stimulation provided by the sites are separated in time by an inter-stimulus interval (“t_{ISI}”). In order that the evoked percept is a continuous motion (rather than discrete percepts), this inter-stimulus interval can be maintained less than 500 milliseconds (e.g., less than 400 millisecond).

In another example, if the stimulation provided by two adjacent sites in the sequence overlap by too great a degree, the percept evoked thereby will be perceived as a single, continuous, and non-moving contact or other tactile percept. To avoid this, the stimulation provided by two adjacent sites in the sequence could be non-overlapping, could overlap by less than 25 milliseconds, could overlap with respect to less than two or less than one pulse of stimulation, or could overlap with respect to only one, synchronous pulse of stimulation (corresponding to an inter-stimulus interval of 0, within the meaning of FIG. 1G).

The perceived speed of such a motion could be controlled in a variety of ways. In some examples, the perceived speed could be increased by increasing a distance between the perceptual fields of adjacent sites in the sequence. In another example, the perceived speed could be increased by reducing the overall duration of the stimulation provided by all of the sites in the sequence. For a set total stimulation duration across the sites, the speed could be increased by decreasing the relative duration of the stimulation provided by an initial site relative to the stimulation provided by a subsequent site. To illustrate this, FIGS. 1H and 1I depicts respective sets of two stimuli (solid line and dashed line) that could be delivered via respective first and second stimulation sites in a sequence. Providing the set of stimuli in FIG. 1H (with the relative duration of the stimulation from the first site being less) will result in the perception of a higher speed of motion that providing the stimuli in FIG. 1I (with the relative duration of the stimulation from the first site being more).

Evoking the perception of motion in this manner could be performed as a result of detecting that a user's natural hand, a prosthetic hand, or some other body part has come into contact with an object that is moving along the body part (e.g., by detecting motion of the contact with the object from a field of forces detected by an array of force sensors on the body part, by detecting motion from an optical flow detector, directional deformation sense, vibration sensor, or other motion sensor). Additionally or alternatively, the perception of motion could be evoked to provide sensation related to a simulated object (e.g., in a virtual environment, that has come into contact and is moving along the surface of a user's virtual hand), to provide an element of a user interface (e.g., to provide an indication of change, and optionally the degree and direction of change, of a virtual indicator), or to present abstract information to a user.

In yet another example, a perception of the concavity (or convexity) of an object can be evoked by simulating the timing and magnitude of delivered stimulation across three (or more) sites along the direction of concavity (or convexity). That is, since contact with a convex object occurs “middle first,” and the force exerted thereby is greatest in the middle, the perception of such an event can be evoked by providing stimulation to a ‘middle’ site of three or more sites prior to beginning to deliver stimulation via two or more ‘outer’ sites, and also by providing higher-magnitude stimulation to the middle site relative to the outer sites. Such a process, for both the contact and eventual removal, of a convex object is illustrated in FIG. 1J, with the solid line representing the time-varying stimulus profile delivered via a middle site and the dashed line representing the time-varying stimulus profile delivered via two or more outer sites. Conversely, the perception of contact with a convex object could be evoked by delivering the dashed line time-varying stimulus profile delivered via the middle site and the solid line time-varying stimulus profile delivered via the outer sites.

As another example, the systems and methods described herein could be used to deliver tactile indications of letters, symbols, or other abstract shapes. This can be accomplished by providing a spatiotemporal pattern of stimulation sequentially across a set of stimulation sites whose perceptual fields correspond to the commanded shape (e.g., letter). As an illustrative example, FIG. 1K depicts a set of stimulation sites through which stimulation could be provided in sequence (e.g., according to the sequence indicated by the arrows) in order to indicate a capital letter “A.” Providing stimulation in such a sequenced manner (e.g., such that the perception is one of a patterned motion, by controlling the inter-stimulus interval to be non-negative and less than 500 milliseconds), the shape of the indicated symbol can be more accurately perceived then, e.g., providing simultaneous (e.g., synchronized) stimulation through the same set of stimulation sites.

### II. Example Systems

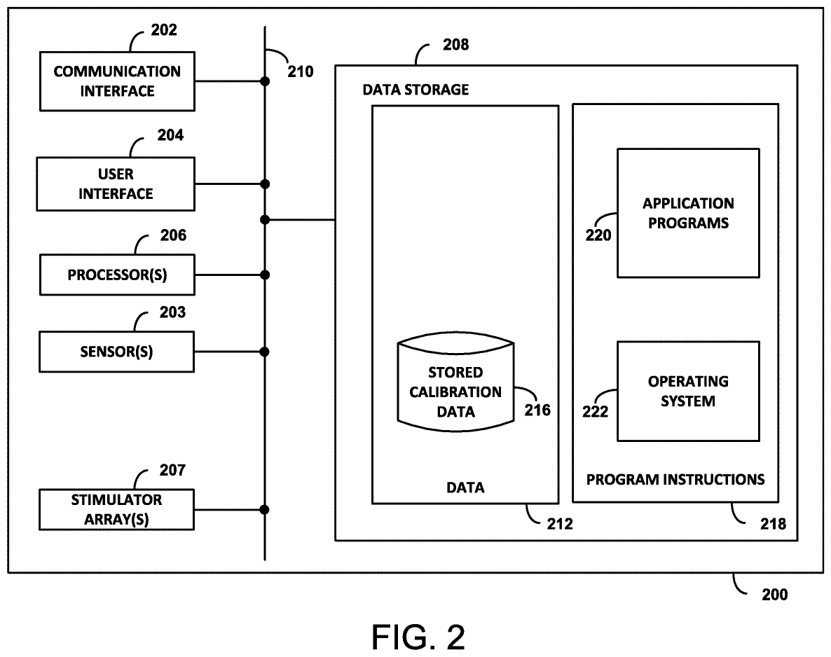

FIG. 2 illustrates an example system 200 that may be used to implement the methods and/or systems described herein. By way of example and without limitation, system 200 may be or include a computer (such as a desktop, notebook, tablet, or handheld computer, a server), elements of an implanted system, elements of a prosthetic device, elements of an assistive device, or some other type of device or system or combination of devices and/or systems (e.g., a prosthetic hand, an implanted stimulator device, and a tablet or other portable device for communicating with and operating the hand and stimulator, e.g., via wired and/or wireless communications links). It should be understood that elements of system 200 may represent a physical instrument and/or computing device such as a server, a particular physical hardware platform on which applications operate in software, or other combinations of hardware and software that are configured to carry out functions as described herein.

As shown in FIG. 2, system 200 may include a communication interface 202, one or more sensors 203 (e.g., force sensors), a user interface 204, one or more processors 206, one or more multichannel arrays 207, and data storage 208, all of which may be communicatively linked together by a system bus, network, or other connection mechanism 210.

Communication interface 202 may function to allow system 200 to communicate, using analog or digital modulation of electric, magnetic, electromagnetic, optical, or other signals, with other devices (e.g., with databases that contain sets of training inputs or related data, e.g., map data that can be updated based on additional user inputs), access networks, and/or transport networks. Thus, communication interface 202 may facilitate circuit-switched and/or packet-switched communication, such as plain old telephone service (POTS) communication and/or Internet protocol (IP) or other packetized communication. For instance, communication interface 202 may include a chipset and antenna arranged for wireless communication with a radio access network or an access point. Also, communication interface 202 may take the form of or include a wireline interface, such as an Ethernet, Universal Serial Bus (USB), or High-Definition Multimedia Interface (HDMI) port. Communication interface 202 may also take the form of or include a wireless interface, such as a WiFi, BLUETOOTH®, global positioning system (GPS), or wide-area wireless interface (e.g., WiMAX, 3GPP Long-Term Evolution (LTE), or 3GPP 5G). However, other forms of physical layer interfaces and other types of standard or proprietary communication protocols may be used over communication interface 202. Furthermore, communication interface 202 may comprise multiple physical communication interfaces (e.g., a WiFi interface, a BLUETOOTH® interface, and a wide-area wireless interface).

In some examples, communication interface 202 could be used to communicate with one or more of sensors (e.g., of sensors 203), actuators (e.g., actuators of a prosthetic hand, wheelchair, or other prosthetic or assistive device), arrays of sensing electrodes or other biosignal sensing components (e.g., an array of penetrating electrodes to detect activity in motor cortex), and/or multichannel arrays (e.g., of channels 207) that are physically separate from the system 200 (e.g., that are part of a separate system, which may include its own processors, communications interface, etc.) to allow the system 200 to control and/or communicate with such separate elements, e.g., to implement a method as described herein. For example, a force, edge, motion, vibration, or other tactile sensor that is part of a prosthetic hand that includes a wireless communication functionality could be operated by the system 200 using the communication interface 202 and/or the system 200 could receive force sensor, edge sensor, motion sensor, texture sensor, or other tactile sensor data from such a sensor using the communication interface 202. In another example, a multichannel array that is part of an implanted stimulator/biosignal sensor system that includes a wireless communication functionality could be operated by the system 200 using the communication interface 202 and/or the system 200 could transmit time-varying stimulus profile data or other stimulus information to such a stimulator system using the communication interface 202.

User interface 204 may function to allow system 200 to interact with a user, for example to receive input from and/or to provide output to the user. Thus, user interface 204 may include input components such as a keypad, keyboard, touch-sensitive or presence-sensitive panel, computer mouse, trackball, joystick, microphone, and so on. User interface 204 may also include one or more output components such as a display screen which, for example, may be combined with a presence-sensitive panel. The display screen may be based on CRT, LCD, and/or LED technologies, or other technologies now known or later developed. User interface 204 may also be configured to generate audible output(s), via a speaker, speaker jack, audio output port, audio output device, earphones, and/or other similar devices. The user interface 204 may be operable to permit a user to program stimulation profiles (e.g., to determine calibration data for the system), to program sets of channels of the multichannel array 207 to operate to provide synchronous or other spatiotemporally patterned stimulation, or to perform some other experimental or clinical operation.

Processor(s) 206 may comprise one or more general purpose processors—e.g., microprocessors—and/or one or more special purpose processors—e.g., digital signal processors (DSPs), graphics processing units (GPUs), floating point units (FPUs), network processors, tensor processing units (TPUs), or application-specific integrated circuits (ASICs). Data storage 208 may include one or more volatile and/or non-volatile storage components, such as magnetic, optical, flash, or organic storage, and may be integrated in whole or in part with processor(s) 206 and/or with some other element of the system (e.g., programmable switches of the multichannel array(s) 207 that can be operated to specify sets of two or more channels of the array via which to provide stimulus synchronously). Data storage 208 may include removable and/or non-removable components.

Processor(s) 206 may be capable of executing program instructions 218 (e.g., compiled or non-compiled program logic and/or machine code) stored in data storage 208 to carry out the various functions described herein. Therefore, data storage 208 may include a non-transitory computer-readable medium, having stored thereon program instructions that, upon execution by system 200, cause system 200 to carry out any of the methods, processes, or functions disclosed in this specification and/or the accompanying drawings. The execution of program instructions 218 by processor(s) 206 may result in processor 206 using data 212.

By way of example, program instructions 218 may include an operating system 222 (e.g., an operating system kernel, device driver(s), and/or other modules) and one or more application programs 220 (e.g., functions for executing the methods described herein) installed on system 200. Data 212 may include stored calibration data 216 (e.g., stored sets of two or more channels whose locations of evoked stimulus on the user's body at least partially overlap, stored stimulus profiles determining how to map time-varying input force signals into time-varying stimulus profiles to be delivered synchronously to sets of channels, stored information about the pattern and location of stimulus when evoked by stimulating each electrode as perceived on a user's skin) that can be used to determine how to operate the multichannel array(s) 207 provide sensory stimulus to a user based on force, edge, motion, texture, or other tactile signal(s) detected using the sensors(s) 203.

Application programs 220 may communicate with operating system 222 through one or more application programming interfaces (APIs). These APIs may facilitate, for instance, application programs 220 transmitting or receiving information via communication interface 202, receiving and/or displaying information on user interface 204, and so on.

Application programs 220 may take the form of “apps” that could be downloadable to system 200 through one or more online application stores or application markets (via, e.g., the communication interface 202). However, application programs can also be installed on system 200 in other ways, such as via a web browser or through a physical interface (e.g., a USB port) of the system 200.

Sensor(s) 203 may include one or more flex sensors, pressure sensors, displacement sensors, edge sensors, texture sensors, slip sensors, motion sensors, vibration sensors, optical flow sensors, accelerometers, gyroscopes, limit switches, temperature sensors, or other sensors configured to detect force or other tactile information (e.g., force exerted onto a specified location of a prosthetic hand, a shape of an edge or other information about the geometry of an object contacting the prosthetic hand, the motion of an object in contact with the prosthetic hand relative to a surface of the prosthetic hand). Sensor(s) 203 may be implanted and/or wearable in order to measure forces, motions, edges, or other tactile energies exerted onto a user's natural hand (e.g., to restore force or other tactile sensation for users who have experienced a spinal cord injury).

Multichannel array(s) 207 may include arrays of electrodes, light emitters, or other elements to controllably generate currents, lights, fields, mechanical forces, or other energies sufficient to excite cells of the somatosensory cortex. The channels of the multichannel array(s) 207 may operate by penetrating the somatosensory cortex (e.g., to deliver electrical, optical, or other stimulatory energies to a specific layer within the cortex) and/or may operate from the surface of the cortex (e.g., to emit optical energy down into the cortex to a specified layer that includes cells that have been genetically modified to be receptive to such optical signals).

### III. Example Methods

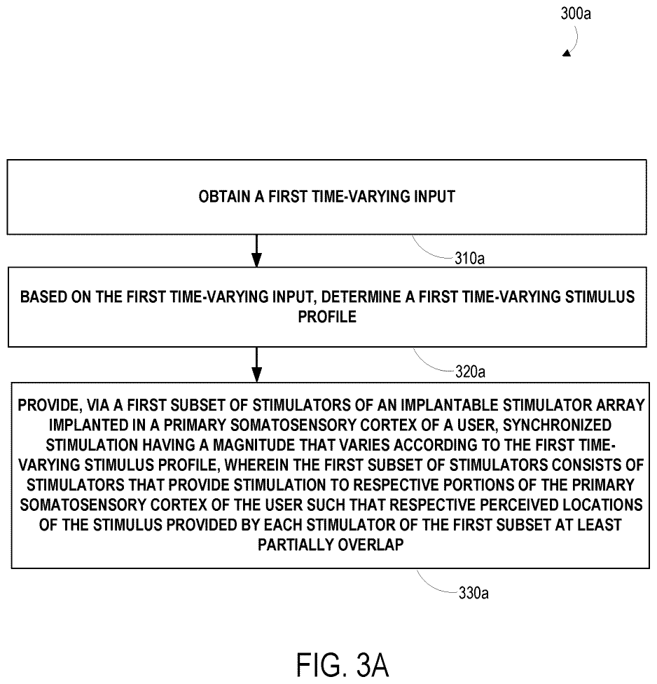

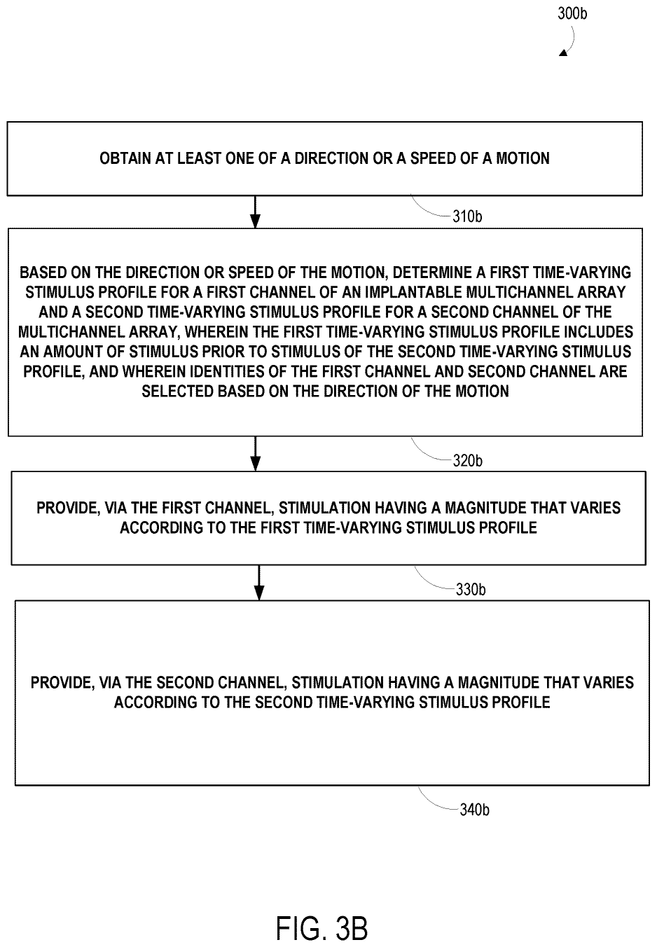

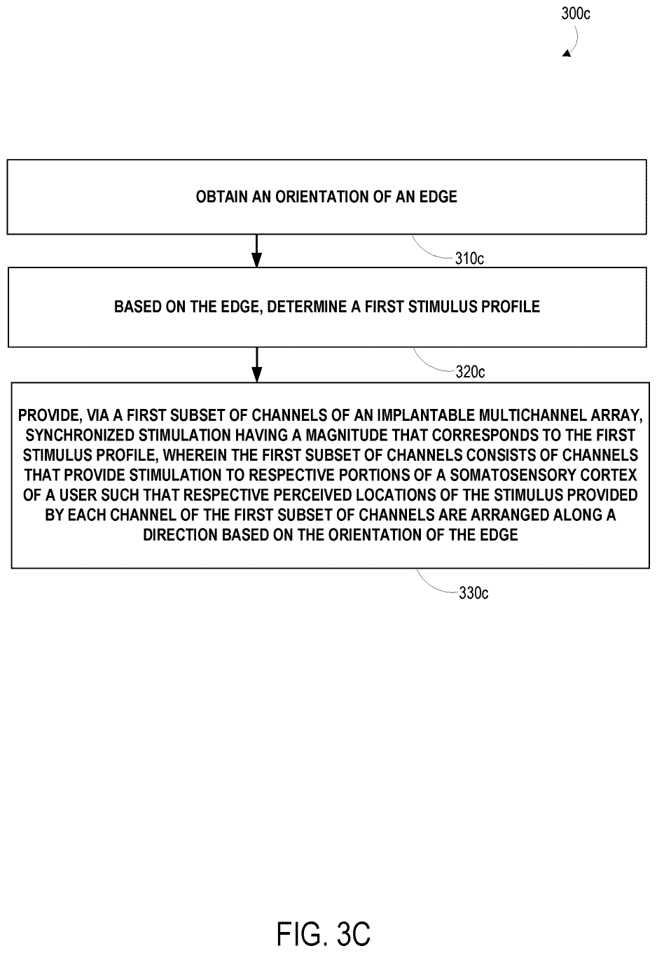

FIG. 3A depicts an example method 300a. The method 300a includes obtaining a first time-varying input (310a). The method 300a additionally includes, based on the first time-varying input, determining a first time-varying stimulus profile (320a). The method 300a also includes providing, via a first subset of channels of an implantable multichannel array implanted in a somatosensory cortex of a user, synchronized stimulation having a magnitude that varies according to the first time-varying stimulus profile, wherein the first subset of channels consists of channels that provide stimulation to respective portions of the somatosensory cortex of the user such that respective perceived locations of the stimulus provided by each channel of the first subset of channels at least partially overlap (330a). The method 300a could include additional steps or features.

FIG. 3B depicts an example method 300b. The method 300b includes obtaining at least one of a direction or a speed of a motion (310b). The method 300b also includes, based on the direction or speed of the motion, determining a first time-varying stimulus profile for a first channel of an implantable multichannel array and a second time-varying stimulus profile for a second channel of the multichannel array, wherein the first time-varying stimulus profile includes an amount of stimulus prior to stimulus of the second time-varying stimulus profile, and wherein identities of the first channel and second channel are selected based on the direction of the motion (320b). The method 300b yet further includes providing, via the first channel, stimulation having a magnitude that varies according to the first time-varying stimulus profile (330b). The method 300b also includes providing, via the second channel, stimulation having a magnitude that varies according to the second time-varying stimulus profile (340b). The method 300b could include additional steps or features.

FIG. 3C depicts an example method 300c. The method 300c includes obtaining an orientation of an edge (310c). The method 300c also includes, based on the edge, determining a first stimulus profile (320c). The method 300c yet further includes providing, via a first subset of channels of an implantable multichannel array, synchronized stimulation having a magnitude that corresponds to the first stimulus profile, wherein the first subset of channels consists of channels that provide stimulation to respective portions of a somatosensory cortex of a user such that respective perceived locations of the stimulus provided by each channel of the first subset of channels are arranged along a direction based on the orientation of the edge (330c). The method 300c could include additional steps or features.

It should be understood that arrangements described herein are for purposes of example only. As such, those skilled in the art will appreciate that other arrangements and other elements (e.g. machines, interfaces, operations, orders, and groupings of operations, etc.) can be used instead of or in addition to the illustrated elements or arrangements.

### IV. Experimental Results

Manual interactions with objects are supported by tactile signals from the hand. This tactile feedback can be restored in brain-controlled bionic hands via intracortical microstimulation (ICMS) of somatosensory cortex (S1), for example, using the embodiments described herein. In ICMS-based tactile feedback, contact force can be signaled by modulating the stimulation intensity based on the output of force sensors on the bionic hand or other sources (e.g., a force sensor implanted sub-dermally in the hand of a person who has suffered a spinal cord injury), which in turn modulates the perceived magnitude of the sensation. In the present study, we gauged the dynamic range and precision of ICMS-based force feedback in three human participants implanted with multichannel arrays of microelectrodes in S1. To this end, we measured the increases in sensation magnitude resulting from increases in ICMS amplitude and participant's ability to distinguish between different intensity levels. We then assessed whether we could improve the fidelity of this feedback by implementing “biomimetic” ICMS-trains, designed to evoke patterns of neuronal activity that more closely mimic those in natural touch, and by delivering ICMS through multiple channels at once. We found that multi-channel biomimetic ICMS gives rise to stronger and more distinguishable sensations than does its single-channel counterpart. We conclude that multi-channel biomimetic ICMS conveys finely graded force feedback that more closely approximates the sensitivity conferred by natural touch.

Manual interactions with objects rely critically on tactile signals from the hand. To be useful, tactile feedback needs to convey information about contact events, including information about contact location and force. Information about location can be intuitively conveyed by matching force sensors on the bionic hand (or other sources of force data) with somatotopically appropriate electrodes in S1. For example, a force sensor on the index fingertip of the bionic hand drives stimulation of electrodes located in the index representation of S1, thus producing a sensation experienced on the index finger. Information about contact force can be conveyed by modulating ICMS amplitude according to the output of the sensor, where higher stimulation amplitudes give rise to more intense touch sensations, paralleling the sensory correlates of increases in force on the skin.

The objective of the present study was to examine the precision and accuracy of ICMS-based tactile feedback about contact force in three human participants implanted with arrays of microelectrodes in S1. To this end, we first characterized the increases in sensation magnitude resulting from increases in ICMS amplitude and gauged the intensity of these percepts against tactile benchmarks. We found that the intensity of ICMS-evoked sensations was highly electrode dependent and often faint. Furthermore, only a few discriminable levels of intensity could be achieved using standard force feedback, which consists of linearly modulating ICMS amplitude according to the applied force. Seeking to improve the range and precision of ICMS-based force feedback, we implemented “biomimetic” ICMS-trains, which, by emphasizing contact transients and de-emphasizing maintained contact, evoke patterns of neuronal activity that more closely mimic those in natural touch. We found that biomimetic ICMS yielded higher resolution force feedback than did its linear counterpart, even though the total injected charge of biomimetic ICMS spanned a narrower range. Next, we investigated whether we could further improve force feedback by delivering biomimetic ICMS through multiple electrodes simultaneously. We found that multi-channel biomimetic ICMS gives rise to stronger and more distinguishable sensations than does its single-channel counterpart, thus enabling precise force feedback over a wider range of forces. We conclude that biomimetic, multi-channel ICMS more closely approximates the sensitivity conferred by natural touch than does linear or biomimetic ICMS delivered through a single electrode

### Results

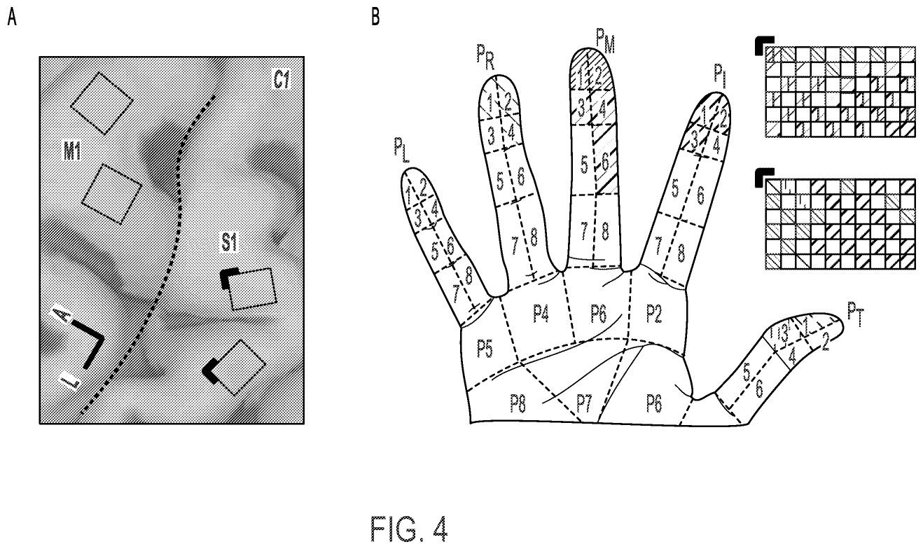

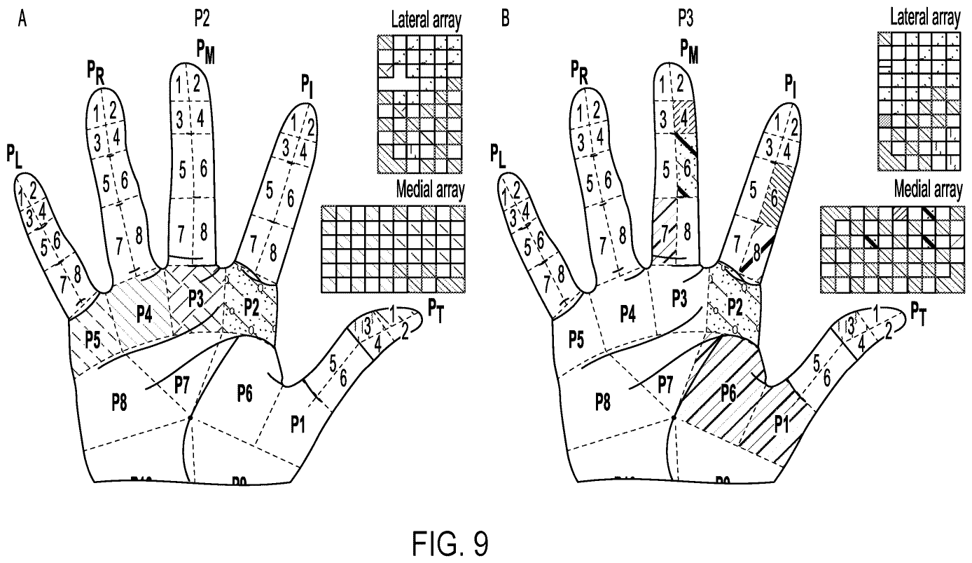

Three participants with cervical spinal cord injury were each implanted with four electrode arrays, two in the arm and hand representation of motor cortex and two in the hand representation in Brodmann's area 1 of S1 (the arrangement of the electrodes is depicted in FIG. 4A). In all three participants, stimulation through electrodes in S1 evoked sensations experienced on the contralateral hand, following the expected somatotopic organization (FIG. 4B for C1, FIG. 9A for P2, and FIG. 9B for P3).

FIG. 4A depicts the locations of implantation of four Utah arrays (Blackrock Neurotech, Inc.) in participant C1, two in the hand representation of S1 based on localization with fMRI. FIG. 4B depicts the locations of the projected fields (the locations on the hand where sensations are experienced) for each S1 channel for participant C1. The top array is medial, the bottom lateral, with the black corners indicating alignment.

FIG. 9A depicts the locations of the projected fields (the location on the hand where sensations are experienced) for each S1 channel for participant P2. FIG. 9B depicts the locations of the projected fields for each S1 channel for participant P3. The top array is medial, bottom one lateral. Colors denote the location of the projected field. Gray squares denote electrodes that evoked sensations on the dorsum of the hand, and white squares denote unwired electrodes.

### The Dynamic Range of ICMS-Evoked Sensations is Electrode Dependent

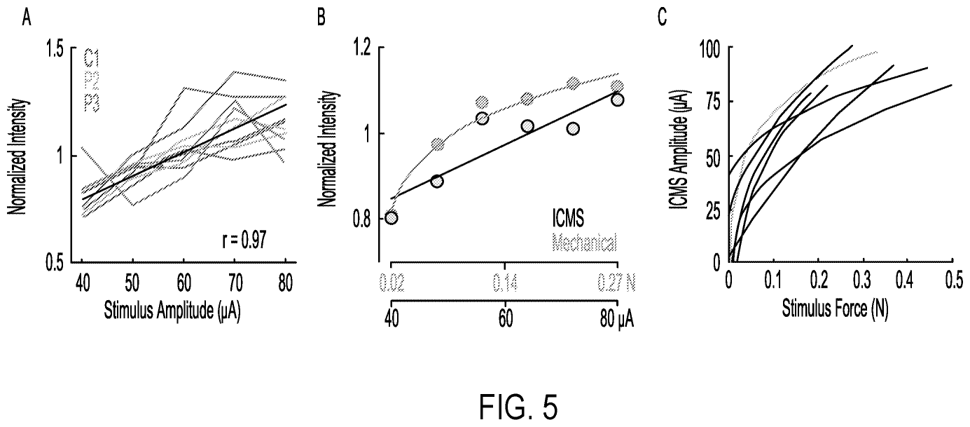

In natural touch, increases in contact force lead to increases in the firing rate of active neurons and in the recruitment of additional neurons in a somatotopically determined region of S1. Increasing the ICMS amplitude leads to the same qualitative pattern of neuronal activation and to increases in the perceived magnitude of the evoked sensation, analogous to the sensory correlates of increases in pressure. With this in mind, we gauged the consistency of this relationship across participants and stimulating electrodes. The three participants rated the perceived magnitude of a sensation on a numerical scale of their choosing. The ratings were then normalized by the mean rating for each electrode and their relationship with ICMS amplitude was characterized. On every electrode tested, perceived magnitude increased approximately linearly with ICMS amplitude, replicating previous findings (FIG. 5A). The median correlation between amplitude and intensity rating was 0.97, with all but two correlations (both from participant P3) above 0.9 (range: 0.47 to 0.99). The relationship between ICMS amplitude and perceived magnitude is thus robust.

FIG. 5A depicts normalized magnitude ratings following ICMS through single channels for 3 participants. Each line denotes ratings for one channel, different colors denote different channels and participants. FIG. 5B depicts normalized ratings when ICMS and mechanical stimuli are interleaved. FIG. 5C depicts equal intensity contours for ICMS-evoked and mechanically evoked sensations. Each line represents the contour derived from the ratings from one stimulating channel. The teal line corresponds to the contour of the channel shown in FIG. 5B. while perceived magnitude increases with amplitude on all channels, the magnitude of the sensations varies widely across channels.

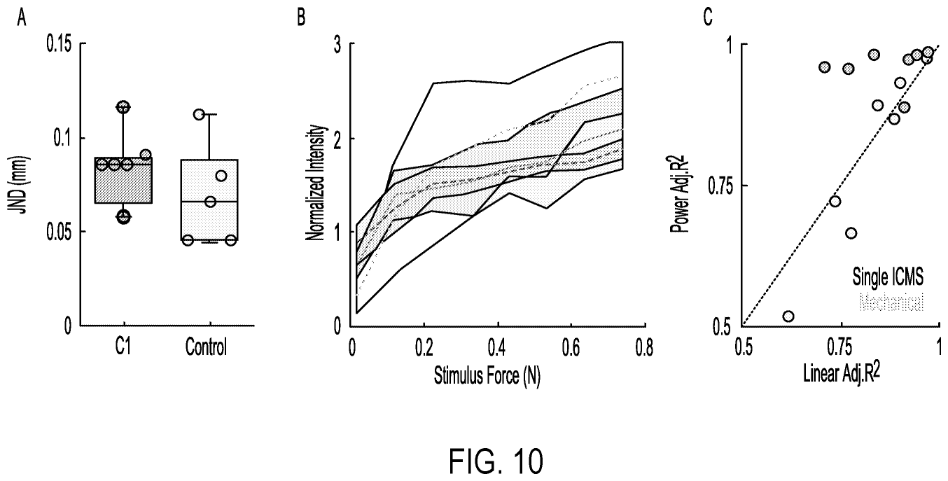

The limitation of the magnitude estimation approach, however, is that ratings are (by virtue of the paradigm) participant-specific, so they cannot be benchmarked to natural touch. Furthermore, ratings are normalized within each block or session so ratings cannot be compared across sessions. To overcome these limitations, we leveraged the fact that participant C1's tactile sensation on the hand is on par with that of able-bodied controls (FIGS. 10A and 10B). Participant C1 judged the magnitude of the sensations evoked by ICMS trains and by skin indentations (delivered to a location matching the projected field of the stimulating electrode) of varying force, with the two types of stimuli interleaved randomly within each experimental block. We could then directly compare the magnitude of ICMS-evoked and mechanically evoked sensations (FIG. 5B) because both stimulus types were rated on the same scale. Furthermore, assuming the perceived magnitude of the tactile stimuli remained constant across sessions, we could also compare the perceived magnitude of ICMS delivered through different electrodes on different sessions (FIG. 10B). From these combined ICMS and touch sessions, we constructed equal intensity contours for the two stimulus types (FIG. 5C), allowing us to determine the ICMS amplitude required to evoke a sensation whose intensity corresponds to a given force and vice-versa. First, we found that perceived magnitude increased approximately linearly with ICMS amplitude but as a (decelerating) power function of mechanical amplitude (FIG. 10C). Accordingly, the iso-intensity contours also followed a power law, reflecting the non-linearity in the mechanically evoked sensations (FIG. 5C). Furthermore, while the magnitude of ICMS-evoked sensations increased with amplitude on all electrodes, the intensity range of sensations varied widely across electrodes: some electrodes could only evoke weak sensations (<0.3 N, the weight of a marshmallow balanced on a finger), whereas others could evoke sensations commensurate with moderate forces (~0.5 N, the weight of an egg). Given that contact forces often exceed 1 N during object interactions, the dynamic range of ICMS-evoked sensations, constrained by the maximum safe stimulation level (generally held to be 100 μA for a single electrode), is generally narrow.

FIG. 10A depicts the mechanical ‘just noticeable differences’ (JNDs) for participant C1 (each point denotes performance on an individual digit) and for 5 able-bodied controls (each point denotes performance of one participant on the tip of the middle finger). The participant's ability to distinguish the depth of indentation is comparable to that of the controls. FIG. 10B depicts normalized intensity ratings for participant C1 on two different fingers (denoted by different colors). Ratings are consistent across fingers. Note that the normalization was performed based on the grand mean rating, which included ratings of single-channel ICMS stimuli, which tended to be weaker than the mechanical ones. FIG. 10C depicts goodness of fir for ICCMS or mechanical intensity ratings when using power vs. linear functions. Power functions provide better fits for intensity ratings of skin indentations buy not ICMS.

### The Discriminability of Evoked Percepts is Electrode Dependent

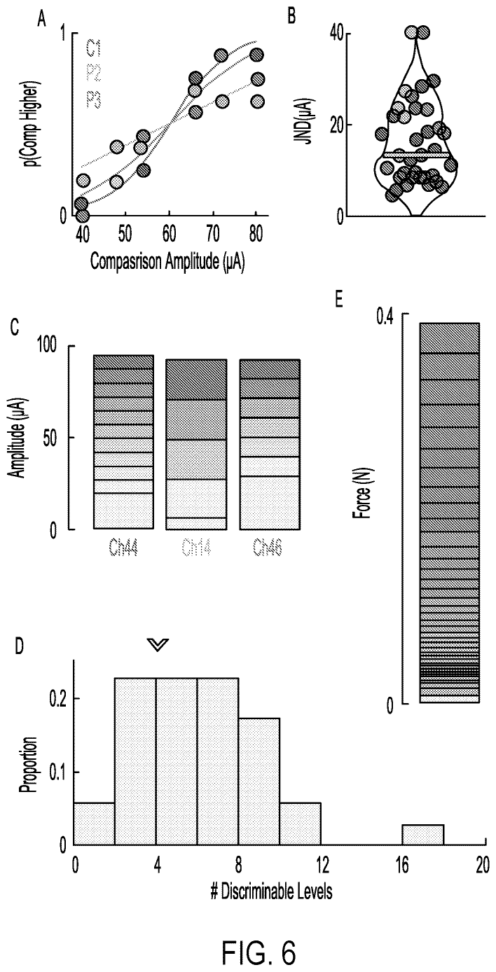

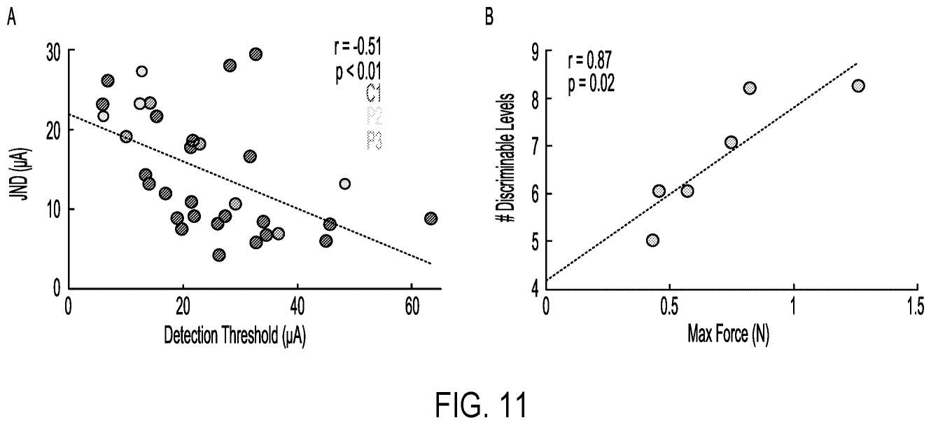

Having established that the mapping between ICMS amplitude and perceived contact force was electrode dependent, we investigated how sensitive participants were to changes in ICMS amplitude. To this end, we had participants discriminate flat ICMS trains that varied in amplitude. In brief, we presented two stimuli—a reference stimulus at 60 μA and a comparison whose amplitude varied between 40 and 80 μA—and the participant reported which of the two was more intense. From the behavioral performance, (FIG. 6A), we computed, as an index of sensitivity for each electrode, the Just Noticeable Difference (JND), which denotes the change in stimulus amplitude that would yield a specified level of performance (75% correct). While the median JND was around 13.5 μA (FIG. 6B), consistent with previous results in humans and monkeys, JNDs varied widely across electrodes and participants (interquartile range: 8.5 to 22.9 μA). Having measured the detection threshold and the JND for each electrode, we computed the number of discriminable levels (FIG. 6C), defined as the number of JNDs between detection threshold and maximum safe amplitude (100 μA). Note that ICMS does not follow Weber's law so the JND measured for each electrode at any amplitude applies across the range of amplitudes. We found that the number of discriminable steps ranged from 2 to 17, with a median of 4 (FIG. 6D), implying that the force feedback conveyed by flat ICMS is coarse, allowing for only a handful of discriminable levels within the safe stimulation range. Electrodes with lower detection thresholds tended to have higher JNDs (r=−0.51, p<0.01, FIG. 11A), suggesting that JNDs do not simply reflect overall sensitivity to ICMS. Electrodes with more discriminable levels also tended to evoke the most intense sensations (r=0.87, p=0.02, FIG. 11B), implying that the JND for each electrode corresponds with a change in intensity that is approximately consistent across electrodes, a phenomenon that is non-trivial given that magnitude functions are not systematically predictable from JNDs in natural perception. As a point of comparison, the native touch of both participant C1 and able-bodied controls (n=5) yielded around 45-50 discriminable levels over this span of forces, an order of magnitude more than what could be achieved with single-channel ICMS over the safe range of amplitudes (FIG. 6E).

FIG. 6A depicts psychometric functions from each participant—note that the curve for each participant is not representative of overall performance. FIG. 6B depicts the JND computed from psychometric functions for all electrode across participants (n=35)—the thick grey line denotes the median (13.5 μA). Note that values above 40 μA are set to 40 μA for graphical purposed (n=2). FIG. 6C depicts estimated discriminable levels plots for the electrodes shown in FIG. 6A. The grey section at the bottom of each bar indicates the subthreshold range for that electrode while the height of each subsequent bar is determined by the JND. FIG. 6D depicts the number of discriminable levels across all electrodes and participants. The arrow indicates the median number of discriminable levels (4). FIG. 6E depicts the estimated discriminable levels for tactile stimulation in the same approximate range of forces (0-0.4 N). Note that the JNDs increase for higher force levels following Weber's law.

FIG. 11A depicts how the discriminability of an electrode (JND) is inversely correlated with its detection threshold. FIG. 11B depicts how electrodes that had a broader dynamic range (i.e., a greater maximum equivalent force) tended to yield more discriminable levels.

### Biomimetic Feedback Confers Greater Sensitivity to Force Changes

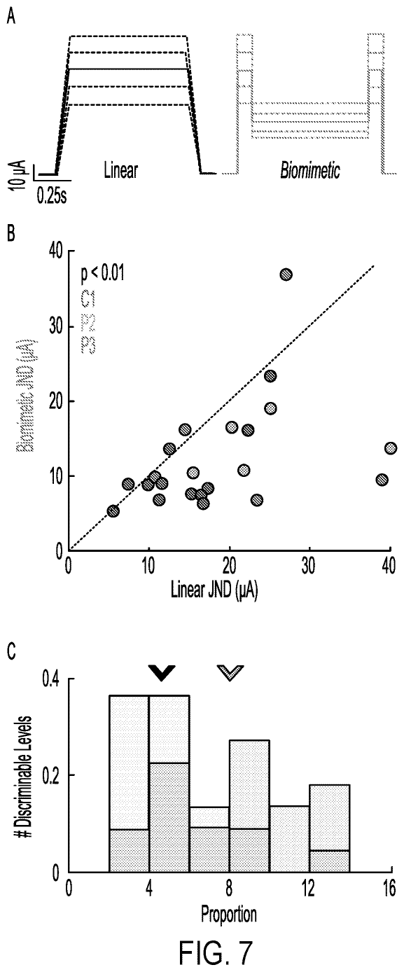

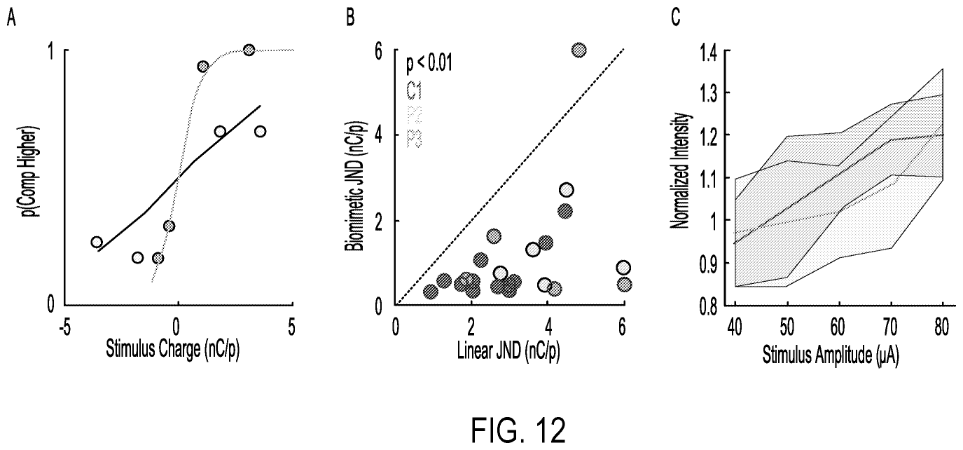

In the experiments described above, ICMS consisted of pulse trains of constant amplitude (flat trains). Such pulse trains typically evoke an abrupt rise in the activation of neurons around the electrode, followed by a slow decrease. In contrast, interactions with objects evoke phasic responses at the onset and offset of contact and much weaker responses (<10%) during maintained contact, a property inherited from the periphery. In studies with electrical stimulation of the peripheral nerves, tactile feedback that features greater sensitivity to contact transients (thereby mimicking natural touch) has been shown to confer greater dexterity to myoelectric bionic hands. With this in mind, we assessed participants' ability to discriminate biomimetic ICMS trains, designed to mimic the response to the onset, maintenance, and termination of contact (FIG. 7A). We compared the participants performance with biomimetic trains to that with trains designed to track the forces linearly, which largely resembled flat trains with short on- and off-ramps. For this comparison, the biomimetic and linear trains were matched in peak amplitude; as a result, the charge delivered in a biomimetic train was less than that in a matched linear one (69±7% on average, see the Methods description below). We found that JNDs for biomimetic ICMS were systematically lower than were their linear counterparts (FIG. 7B, medians: 9.7 and 16.6 μA; IQRs: 7.7 to 16.1 μA and 11.5 to 23.5 μA, respectively, Wilcoxon signed-rank test, n=22, z=3.1, p<0.01). The improved sensitivity to biomimetic ICMS is especially surprising given that biomimetic trains contained less charge. For the set of electrodes tested in this comparison, the median number of discriminable levels increased from 4.5 to 8 with biomimetic sensory feedback (FIG. 7C). Biomimetic ICMS trains thus provide higher resolution force feedback, an advantage that is even more pronounced when expressed in terms of charge rather than peak amplitude (FIGS. 12A and 12B) and does not reflect differences in perceived intensity (FIG. 12C).

FIG. 7A depicts example idealized stimulus profiles for linear (where stimulus amplitude scales with force) and biomimetic (where force transients are emphasized) stimuli. FIG. 7B depicts that JNDs are reduced (and sensitivity enhanced) with biomimetic stimuli versus linear ones. FIG. 7C depicts the distribution of the number of discriminable levels computed from JNDs with linear or biomimetic stimuli. The number of levels increases with biomimetic stimuli versus linear ones (median=8 vs. 5, respectively).

FIG. 12A depicts psychometric functions for biomimetic and linear trains as a function of the difference in mean charge per phase between the standard and comparison, for one electrode. FIG. 12B depicts JNDs for biomimetic and linear stimuli across participants and electrodes (n=20). Biomimetic stimuli yield significantly lower JNDs, expressed in terms of charge per phase (Wilcoxon signed-rank test, Z=3.9, p<0.01). FIG. 12C depicts normalized intensity ratings for one channel. Linear ICMS does not give rise to significantly more intense sensations than does biomimetic ICMS when comparing stimuli with the same maximum amplitude. The mean relative intensity was 92+/−3% (n=5 electrodes).

### Multi-Channel Feedback Confers Greater Dynamic Range and Change Sensitivity

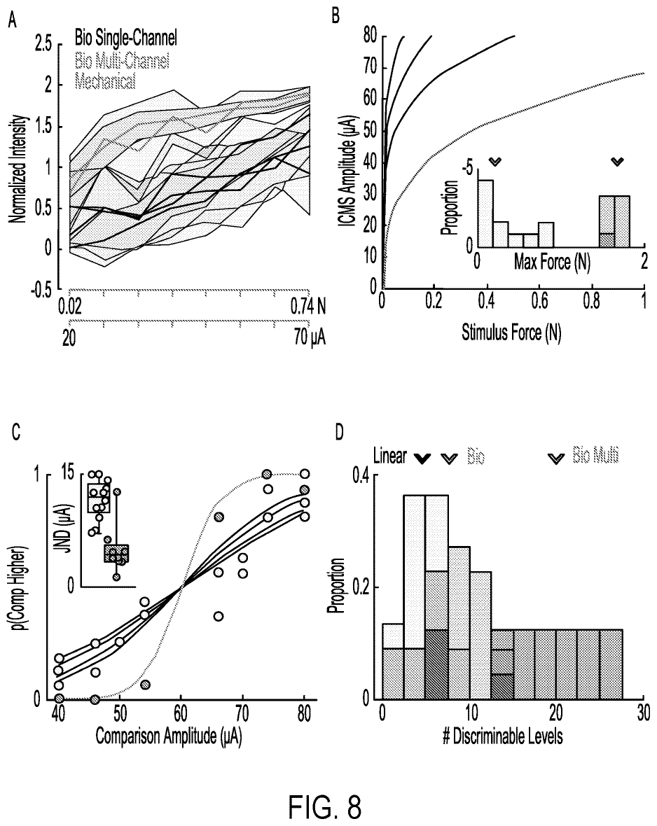

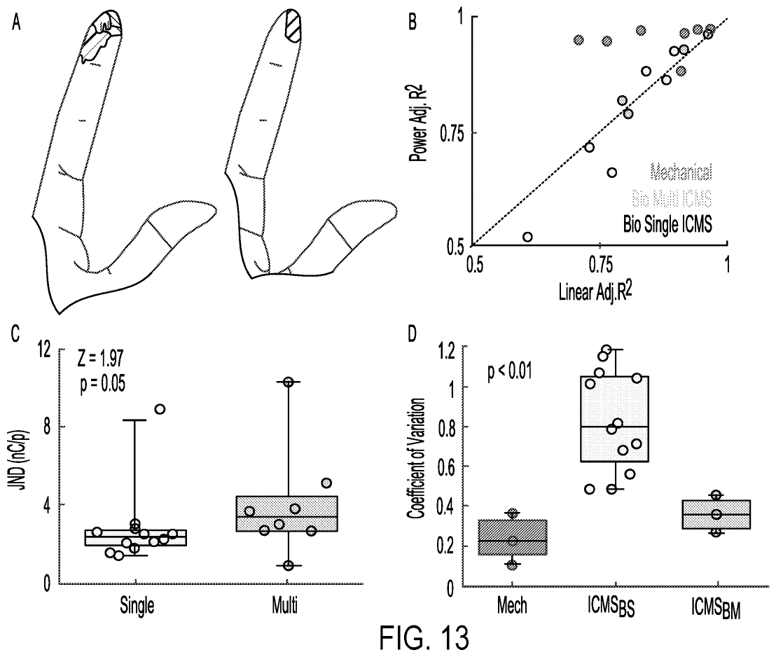

Having characterized the dynamic range and resolution of ICMS-based force feedback delivered through single-channel at a time, we next examined the magnitude of sensations evoked when ICMS was delivered through multiple electrodes simultaneously. For these experiments, we selected groups of 4 electrodes (quads) with overlapping or adjacent projected fields (the hand regions over which the sensations were experienced) (FIG. 13A). That is, when stimulation was delivered through each of the four electrodes individually, the sensation was experienced on an overlapping patch of skin or, in one case, on adjacent digits. In these experiments, ICMS was always biomimetic, comprising higher amplitude phasic stimulation at the onset and offset and weaker stimulation between the two phasic components. First, we assessed whether multi-channel stimulation increased the dynamic range of the evoked sensations by comparing magnitude estimates of intensity with (biomimetic) single-vs. multi-channel stimulation (3 sets of 4 electrodes in participant C1). We found that multi-channel stimulation evoked systematically more intense sensations than did single-channel stimulation when equating the current delivered through each electrode individually (so quad stimulation delivered four times more total current than did its matched single-electrode counterpart, FIG. 8A). Nonetheless, within the safe range of ICMS amplitudes, multi-channel ICMS allowed for a much wider dynamic range than did any electrode in isolation (more than twice the average electrode, FIG. 8B). Indeed, the peak equivalent force reached 2 N, approximately the weight of a mobile phone. Furthermore, the multi-channel ICMS amplitude intensity function still followed a linear relationship with amplitude (FIG. 13B) so this linear relationship is not an artifact of a narrow range of intensities.

FIG. 8A depicts normalized magnitude ratings when ICMS is delivered through electrodes individually or simultaneously, interleaved with mechanical stimuli for an example quad. All ICMS trains were biomimetic. Lines denote the mean while shaded areas denote the standard deviation. FIG. 8B depicts iso-intensity contours for single-channel (gray) and multi-channel (red) ICMS for an example quad. Inset: maximum achievable force for single or multi-channel stimulation across all tested quads, extrapolating fits to 100 μA. FIG. 8C depicts psychometric functions for one quad of electrodes with the performance of the individual electrodes shown in gray and that of the quad in red. Inset: JNDs for all single electrodes and quads tested. The median JND decreased from 12 to 4.3 μA with multi-channel ICMS (Wilcoxon rank sum test: Z=−3.05, p<0.01). FIG. 8D depicts the estimated number of discriminable levels with single-channel ICMS (linear or biomimetic stimuli, median=4.5 and 8, respectively) and multi-channel biomimetic ICMS (median=20). FIGS. 8A and 8B show results from participant C1 while FIGS. 8C and 8D show results from participants C1 and P2.

FIG. 13A depicts projected field locations for individual electrodes individually (left) and simultaneously (right). FIG. 13B shows that linear fits are still equivalent to power law fits for multi-channel stimulation. FIG. 13C depicts discrimination performance for single or multi-channel stimulation when expressed as charge per phase. FIG. 13D depicts how variability of responses is lower for multi-channel stimulation than for single-channel stimulation.

Next, we examined the discriminability of multi-channel biomimetic ICMS trains that varied in amplitude (n=8 quads, 5 from participant C1 and 3 from participant P2). We found that multi-channel ICMS yielded substantially lower JNDs than did its single-channel counterpart (FIG. 8C), mirroring the lower variability in the magnitude estimates of intensity for multi-channel stimulation compared to its single channel counterpart (FIG. 13D). Combined, the wider dynamic range and higher resolution yielded an increase in the median number of discriminable levels from 8 to 20 (FIG. 8D). Thus, biomimetic multi-channel force feedback can yield more precise force feedback over a wider dynamic range than does standard force feedback through a single electrode. Indeed, while JNDs expressed in terms of charge were equivalent or higher for multi-channel than single-channel stimulation (FIG. 13C), the bottleneck in ICMS-based feedback is the charge delivered on any given electrode, which is the primary determinant of stimulation-induced neuronal damage. Multi-channel ICMS circumvents this limitation by distributing charge, thereby increasing both the range and precision of the resulting sensations to a level that more closely approximates natural touch (~20 vs. 45-50 discriminable steps, respectively).

### Discussion

### Biomimetic ICMS Confers Higher Resolution Force Feedback

For ICMS-based feedback, biomimetic trains differing in amplitude are even more discriminable than are their amplitude-matched linear trains. The difference between biomimetic and linear ICMS trains is even more pronounced when the stimulation intensity is expressed in terms of overall charge (FIG. 12B). Biomimetic ICMS-based feedback offers the additional advantage of higher resolution force information, unlike its peripheral counterpart.

### Multi-Channel ICMS Confers Wider Dynamic Range and Higher Resolution

Multi-channel ICMS leads to more intense sensations than single-channel ICMS. This finding is perhaps unsurprising given that multi-channel stimulation entails four times more charge delivery than does single-channel stimulation. In fact, multi-channel stimulation less efficiently modulates the overall perceived magnitude compared to single-channel stimulation when expressed as a function of overall charge (FIG. 13B). The major bottleneck in ICMS-based sensory feedback, however, is that the amplitude used in human experiments is capped at 100 μA, as this level of stimulation has been shown in experiments with monkeys to cause no damage beyond that incurred during implantation. Even if this maximum level turns out to be more conservative than it needs to be, evidence suggests that charge per phase is the main determinant of ICMS-induced neuronal damage. Accordingly, multi-channel ICMS enables a widening of the dynamic range without increasing the charge per phase on any given electrode.

Beyond widening the dynamic range, multi-channel ICMS improved the resolution of the feedback, as gauged by lower JNDs. This improvement is inconsistent with the results of experiments with monkeys, in which multi-channel ICMS yielded similar JNDs as did its single-channel counterpart.

While JNDs are lower for multichannel biomimetic stimulation, each JND leads to a greater increment in perceived magnitude. As a result, while the resolution of multichannel stimulation is higher for ICMS amplitude, it is equivalent for force. Indeed, successive discriminable increments in amplitude are associated with larger increments in force with multichannel stimulation to maintain a correspondence between the level of force exerted and the sensory experience. However, the dynamic range of forces is much higher for multichannel than single channel stimulation.

### Conclusions

Biomimetic multi-channel stimulation doubles the dynamic range of the evoked touch sensations, decreases the JNDs, and yields nearly fivefold more discriminable levels of force than does single-channel linear feedback. Biomimetic trains yield more discriminable percepts and do so more efficiently, in terms of charge. While multi-channel stimulation is not more efficient than is its single channel counterpart with respect to total charge, distributing charge across electrodes increases the dynamic range without increasing the charge density, which is the main determinant of stimulation-induced neuronal damage.

### Methods

### Participants

This study was conducted under an Investigational Device Exemption from the U.S. Food and Drug Administration and approved by the Institutional Review Boards at the University of Pittsburgh and the University of Chicago. The clinical trial is registered at ClinicalTrials.gov (NCT01894802). Informed consent was obtained before any study procedures were conducted. Participant C1 (m), 57 years old at the time of implant, presented with a C4-level ASIA D spinal cord injury (SCI) that occurred 35 years prior to implant. Participant P2 (m), 28 years old at the time of implant, presented with a C5 motor/C6 sensory ASIA B SCI that occurred 10 years prior to implant. Participant P3 (m), 28 years old at the time of implant, presented with a C6 ASIA B SCI that occurred 12 years prior to implant.

### Cortical Implants

We implanted four microelectrode arrays (Blackrock Neurotech, Salt Lake City, UT, USA) in each participant. The two arrays (one medial and one lateral array) in Brodmann's area 1 of somatosensory cortex and were 2.4 mm×4 mm with sixty 1.5-mm long electrode shanks wired in a checkerboard pattern such that 32 electrodes could be stimulated. The two arrays in primary motor cortex were 4 mm×4 mm with one-hundred 1.5-mm long electrode shanks wired such that 96 (C1 and P3) or 88 (P2) electrodes could measure neural activity. The inactive shanks were located at the corners of these arrays. Two percutaneous connectors, each connected to one sensory array and one motor array, were fixed to the participant's skull. We targeted array placement during surgery based on functional neuroimaging (fMRI) of the participants attempting to make movements of the hand and arm (all participants) and imagining feeling sensations on their fingertips (participant P2), within the constraints of anatomical features such as blood vessels and cortical topography.

### Intracortical Microstimulation (ICMS)

Stimulation was delivered via a CereStim 96 (Blackrock Neurotech). Each stimulating pulse consisted of a 200 μs cathodic phase followed by a half-amplitude 400 μs anodic phase (to maintain charge balance), the two phases separated by 100 μs. In all tasks, conditions were interleaved, counterbalanced when appropriate, and randomized within block.

### Multi-Channel ICMS

We selected groups of 4 electrodes, referred to as quads. In most cases, the four electrodes had overlapping projected fields. In one case, one pair of electrodes in the quad were on one digit and the other pair was on the adjacent digit. During an experimental block, we randomly interleaved stimulation through each channel in each quad with stimulation through the entire quad. For multi-channel ICMS, an identical pulse train was delivered through every channel of the quad.

### Mechanical Skin Indentation

To deliver mechanical indentations into the skin of participant C1, we used a V-308 voice coil (Physik Instrumente, USA, MA) to drive an indenter whose tip was 5 mm in diameter. Stimuli were 1 second in duration with 0.1 second ramps, matching the profile of the ICMS, and ranged in amplitude from 0.02 mm to 2 mm. The tip was centered on the location of the projected field for a given electrode as reported by the participant. The indenter tip was then pre-indented into the skin to ensure maintained contact throughout the experimental block. Mechanical indentations were also delivered to five able-bodied participants (all male, 24-33 years of age) under a separate IRB protocol approved by the University of Chicago.

### Amplitude Discrimination

Participants performed an amplitude discrimination task in a two-alternative forced choice paradigm. On each trial, a pair of stimuli, each lasting 1 second, was presented with a 1 second inter-stimulus interval and the participant reported which stimulus was stronger. The reference stimulus, consistent across the experimental block, was paired with a comparison stimulus whose amplitude varied from trial to trial. The order of presentation of the reference and comparison stimuli was randomized and counterbalanced. Data were obtained from each electrode over a minimum of 8 experimental blocks, each consisting of 2 presentations (one for each order) for each stimulus pair.

The frequency of the ICMS stimuli was either 50 Hz (FIG. 6) or 100 Hz (all other experiments). For linear ICMS, two trapezoidal force traces (see force approximation below) were generated (0.1 second ramps and 0.8 second hold) and converted into trains of ICMS that followed the force trace and peaked at an amplitude proportional to the peak force. Biomimetic stimuli consisted of a transient phase-whose duration matched that of the force ramp (0.1 sec) and whose pulse amplitude matched that of the corresponding linear train—and a hold phase during the static component of the force (0.8 sec), during which the pulse amplitude was either 30 μA less than the amplitude in the transient phase or at the detection threshold, whichever was highest. On any given experimental block, the reference amplitude was 60 μA and the comparison stimuli varied in amplitude between 40 and 80 μA (FIG. 7A). Note that the JNDs were the same at 50 and 100 Hz, as has been previously found in experiments with monkeys.

The same paradigm was used to measure amplitude JNDs with the mechanical indenter as for ICMS. The indentation depths varied between 0.85 and 1.15 mm with a standard amplitude of 1 mm and the onset- and offset-ramp speed was 5 mm/s.

### Magnitude Estimation

In this task, participant C1 rated the perceived magnitude of an ICMS train (single- or multi-channel) or mechanical indentation. Briefly, on each trial, the participant was presented with an ICMS or a mechanical stimulus (queued with a fixation cross) and rated its sensory magnitude on a scale of his choosing. If the stimulus was imperceptible, the participant ascribed to it a rating of zero. If one stimulus was twice as intense as another, it was ascribed a rating that was twice as large. The participant was encouraged to use fractions or decimals. At the beginning of each set, the participant was presented with each of the test stimuli in a random order to familiarize them with the stimulus range. The amplitude of the ICMS stimulus varied between 20 and 80 μA while the mechanical stimuli ranged from 0.05 to 2 mm. In all cases the mechanical stimulus was delivered to the location of the projected field for the given electrode. Both ICMS and mechanical stimuli were interleaved throughout each block, with a 5-sec interstimulus interval.

In experimental blocks involving both skin indentations and ICMS, the range of indentation depths was selected in preliminary measurements to match the range of intensities of the electrical stimuli (based on participant ratings) to maximize the overlap and minimize range-related biases. To this end, we estimated, at the start of the session and with the participants help, the range of mechanical indentations that evoked sensations of comparable intensity as their ICMS counterparts (which varied from channel to channel). When multiple channels were tested, we selected the weakest and strongest sensations across channels. We then evenly sampled intermediate indentation depths between these two extremes. In some cases, the intensity of the multi-channel ICMS exceeded the maximum indentation that could be delivered with the indenter, precluding comparisons at higher amplitudes.

A minimum of 8 blocks were completed for each channel and condition. Blocks were sometimes distributed across several days to minimize the effects of adaptation and maintain participant engagement.

### Detection Thresholds

Detection thresholds were measured separately on a quarterly basis as described previously. We either used a 3 up 1 down transformed staircase or the method of limits-both in a 2-alternate forced choice (AFC) design targeting 50% detection performance.

### Projected Fields

Projected fields were collected over multiple years for each electrode and participant. On each trial, a 60-μA, 100-Hz ICMS train was delivered through a given electrode and the participant drew the spatial extent of the sensation on a hand diagram (such as that shown in FIG. 4B) using a tablet. The region enclosed by the drawn boundary constituted an estimate of the projected field for that electrode on that session. Only hand regions that were included in the projected field on two thirds of the sessions were included in the pooled estimate of the projected field.

### Analysis

### Iso-Intensity Contours

From magnitude ratings obtained when both skin indentation and ICMS were interleaved, we fit a power function to the magnitude ratings, Mₘ, for skin indentations of amplitude aₘ:

$${M}_{m} = α {a}_{m}^{γ}$$

and a linear function to the magnitude ratings, Mₑ, of ICMS of amplitude de:

Mₑ=βaₑ

We then derived an equal intensity contour for stimuli of equal perceived magnitude, where Mₘ=Mₑ:

$${a}_{e} = \frac{α}{β} {a}_{m}^{γ}$$

### Force Approximation

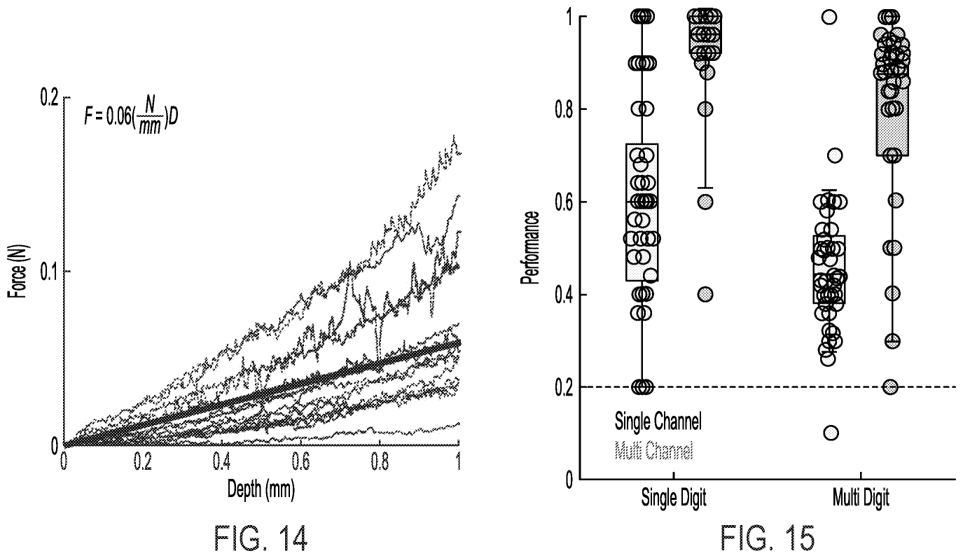

To convert skin indentations into equivalent forces, we measured the relationship between skin indentation and exerted force using a Universal Testing Machine (Instron, FIG. 14). Data was collected from 15 individuals (8 women, 7 men) aged between 21 and 30. We then used this measured function to estimate the forces in the magnitude estimation task.

FIG. 14 depicts indentation depth vs force for 15 human participants using a 2-mm diameter tip with a universal testing machine (Instron). Each line denotes data from one participant, thick black line denotes the mean.

### Psychometric Functions

Psychometric functions were fit with a logistic function:

$$p = \frac{1}{1 + {e}^{k * ( x - μ )}}$$

Where p is the probability of judging the comparison stimulus as more intense than the reference, x is the amplitude, k is the slope, and μ the point of subjective equality (PSE). The just noticeable difference (JND) is half the difference between the amplitude that yields a p of 0.75 and the amplitude that yields a p of 0.25.

### Discriminable Levels

To estimate the number of discriminable levels for ICMS we used the formula:

$$DL = \frac{100 - {dt}_{50}}{JND}$$

where DL is the number of discriminable levels, dt₅₀ is the 50% detection threshold for the channel, and JND is the Just Noticeable Difference for that channel.

To estimate the number of discriminable levels for mechanical stimulation, we computed the Weber fraction from the amplitude discrimination experiment and used a detection threshold of 0.05 μm, estimated from the magnitude estimation experiments. We then extrapolated the discriminable levels until we reached the amplitude whose perceived magnitude matched that of the ICMS at maximum amplitude.

### Contact Localization

Synchronized multi-channel stimulation as described herein also provides benefits with respect to contact localization relative to single-channel stimulation. The circumscription and systematic localization of projected fields (“PFs,” the locations on a person's real or imagined body at/across which stimulation-induced sensations are experienced) can be exploited to convey information about (bionic) hand locations at which contact with an object is established. More focal and thus more easily localizable sensations can be evoked via synchronized multi-channel stimulation as described herein. To verify this effect, force sensors on a bionic hand were mapped to electrodes with corresponding PFs. For example, sensors on the prosthetic thumb tip drove stimulation through electrodes with PFs on the thumb. In some cases, each sensor drove stimulation through a single electrode; in other cases, sensor output drove quads of electrodes whose PFs largely overlapped. For these overlapping-PF channels, the overlapping component of each PF matched the mapped location of the sensor. Different digits were then touched and the (blindfolded) participant reported which finger was touched. In some experimental blocks, only one (prosthetic) digit was touched at a time; in others, one or two digits were touched. The participant (C1) was warned when more than one finger was possible to be touched in a given experimental block. The same experiment was then repeated, except that digit(s) were selected by the experimenter on the experimental computer (rather than by physically touching the bionic hand). Results from these two paradigms-one with and one without the bionic hand-were similar and thus combined. In all conditions (single vs. multi electrode and/or digit), the participant reported the digit(s) with the dominant PF on the majority of trials. Single-electrode stimulation more often failed to elicit a sensation (30% vs 0%) and more often elicited a sensation reported on an incorrect digit than did multi-electrode stimulation (42 vs. 7%, respectively). These results are shown in FIG. 15. Difference in performance for single-vs. multi-electrode stimulation was more pronounced on blocks with multi-digit touches.

### IV. Additional Experimental Results

Intracortical microstimulation (ICMS) of the somatosensory cortex (S1) evokes vivid tactile sensations, the location and properties of which can be systematically manipulated by varying the stimulus electrode or parameters. This phenomenon can be leveraged to convey feedback from a brain-controlled bionic hand about object interactions. However, previous implementations of ICMS have provided an impoverished sense of touch, limiting dexterous manipulations critical to everyday interactions. This disclosure expands the repertoire of ICMS-based artificial touch to include features beyond location and force to confer greater dexterity to brain-controlled bionic hands. Specifically, the embodiments described herein can be employed to convey information about the local geometrical features of objects and about their motion, leveraging understanding of how these sensory features are encoded in S1 and inspired by stimulation designed for other sensory regions.

To accomplish this, the embodiments herein simultaneously delivered spatially-patterned ICMS through multiple electrodes whose projected fields (PFs)—the patch of skin over which the sensation is experienced-were arranged in a line. Unprompted, the participants reported the sensation of an edge. Next, this paradigm was expanded to more complex shapes and found that participants could intuitively perceive arbitrary planar, curved, and 3D shapes. Adding temporal patterning to the paradigms, ICMS was sequentially delivered through electrodes with spatially discontinuous PFs, which induced participants to report a sensation of movement across their skin. By selecting sets of electrodes with different configurations of PFs, the direction and speed of perceived motion across the skin was systematically manipulated. Such principled spatiotemporal patterning of ICMS as described herein can evoke rich and complex sensations to expand the repertoire of artificial touch.

Touch conveys rich information about interactions with objects to effect complex dexterous behaviors. When one touches an object, they rapidly determine whether it's hard or soft, rough or smooth, hot or cold. Furthermore, one identifies any local contours or ridges under their fingertip such as the edges of a button. This information is consolidated into a 3-dimensional representation of the object in a phenomenon known as stereognosis and is an important aspect of object interactions. This is enabled, in part, by neurons in primary somatosensory cortex (S1) that have receptive fields and tuning properties for these features. Similarly, when an object moves across the skin, populations of neurons with adjacent receptive fields are sequentially activated. In both vision and touch, information about motion is extracted from a spatiotemporal pattern of activation across a sensory sheet (in the retina and in the skin, respectively), a process that has been extensively studied in both modalities. In particular, spinal cord injury often disrupts tactile sensation in addition to motor control and results in an inability to dexterously interact with objects.

This embodiments described herein expand the repertoire of touch sensations beyond these two basic features to improve the functionality of bionic hands. The embodiments herein delivered spatially patterned ICMS to evoke sensations on somatotopically linked parts of the hand. In particular, the somatotopic organization of S1 was used to convey information about the locations of object contact on a bionic hand, for instance, a sensor on the bionic thumb was used to drive neurons with a PF on the participant's perceived thumb. Extending this basic property of single-channel PFs, multiple channels were activated simultaneously to cause participants to intuitively perceive these new sensations as edges or shapes commensurate with predictions from combining individual channels' PFs. To add a sense of apparent motion to these percepts electrodes with adjacent PFs were sequentially activated. The perceived direction of the stimuli was determined by the responses of subpopulation of neurons in area 1. Neurons in area 1 organize their inputs to select specific stimulus features, such as (a) contact area, (b) edge orientation, (c) motion across the skin, or (4) direction of movement. These feature detection neurons have large receptive fields, and require inputs to be presented in specific spatial and/or temporal configurations. The experiments described herein demonstrate that participants can be induced to experience the sequential activation of adjacent PFs as a tactile sensation moving across their skin at a speed dependent upon both the distance between the electrodes PFs and the temporal patterning of the ICMS. Findings reported herein show that spatiotemporal patterning of ICMS can expand the repertoire of artificial touch, thereby enhancing its ability to support dexterous object interactions.

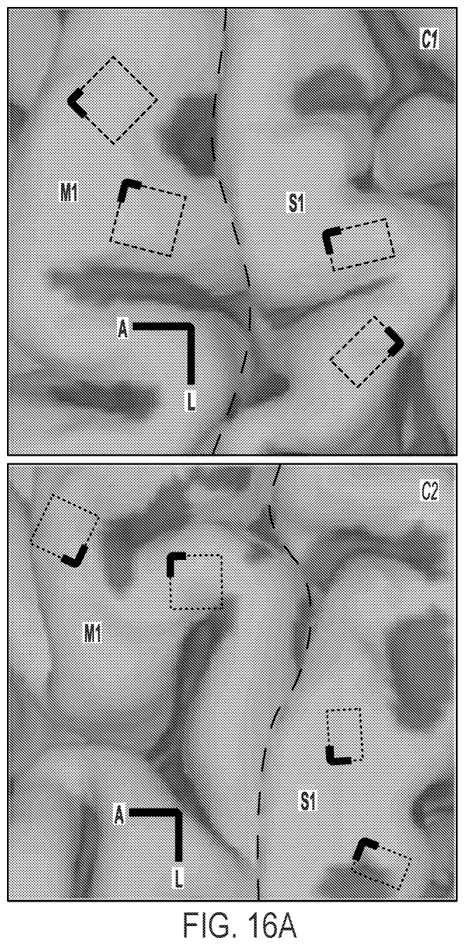

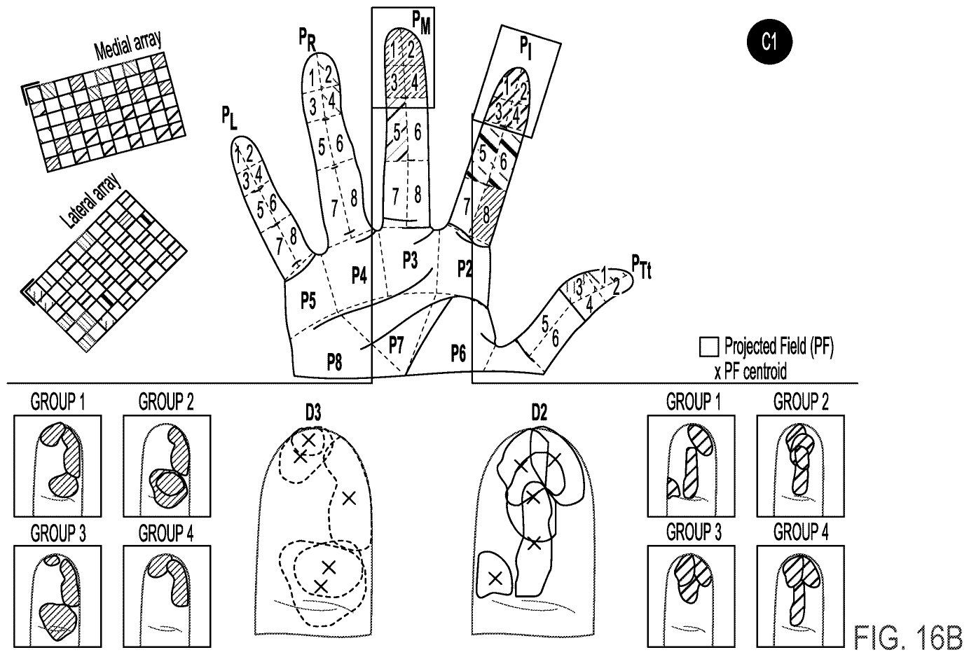

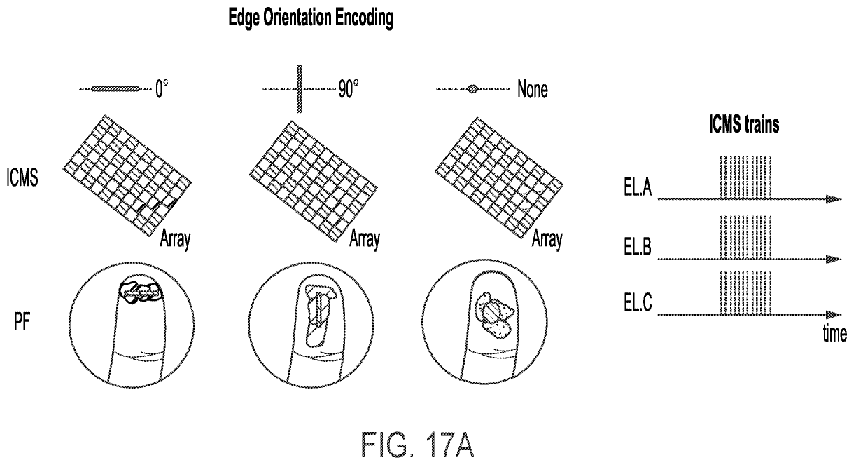

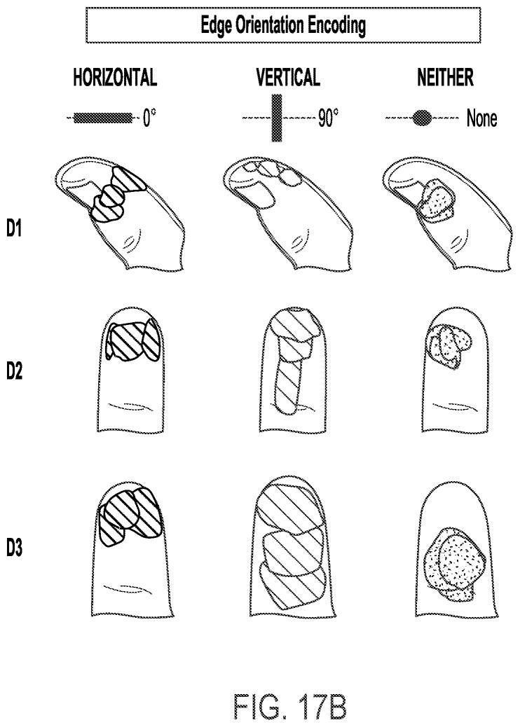

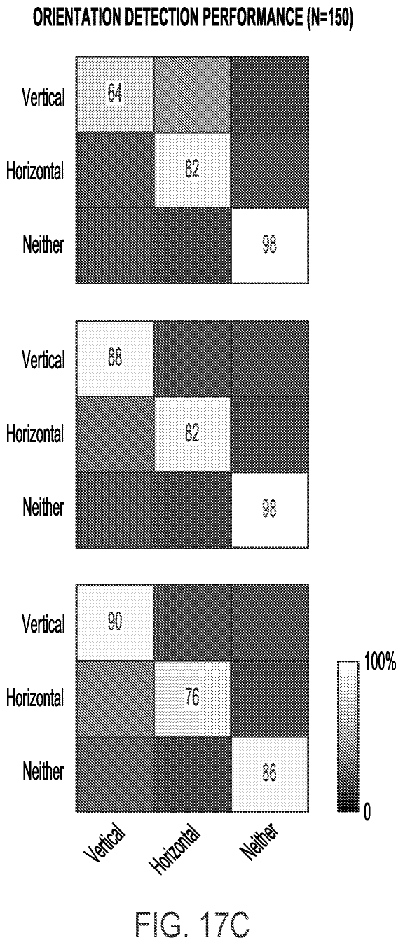

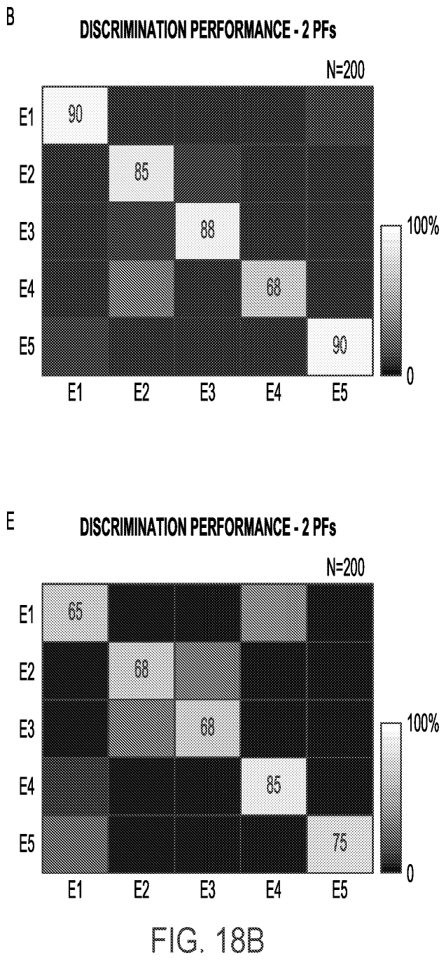

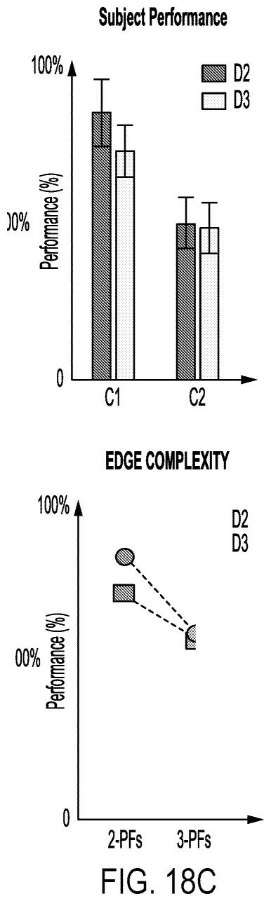

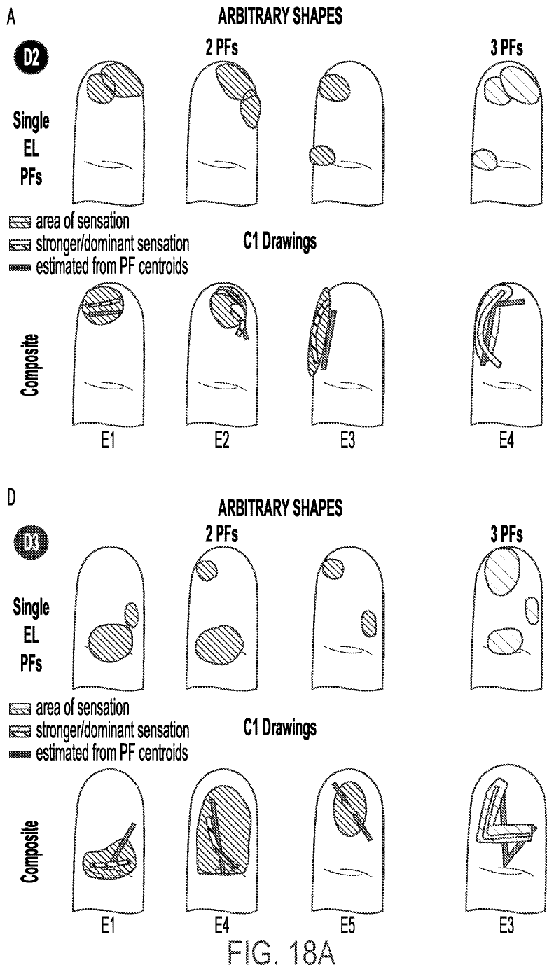

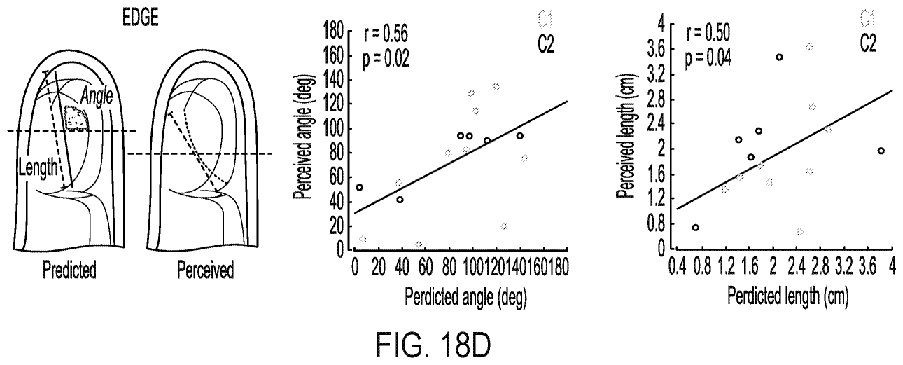

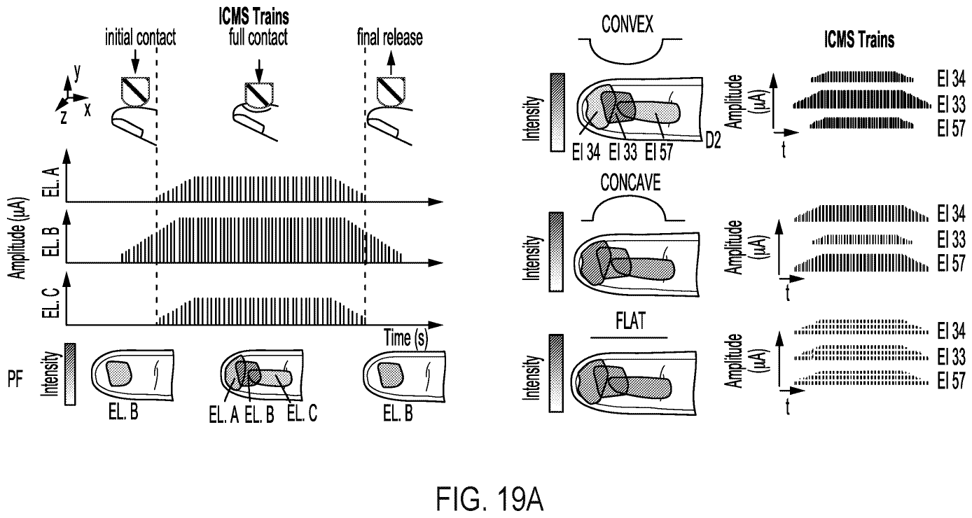

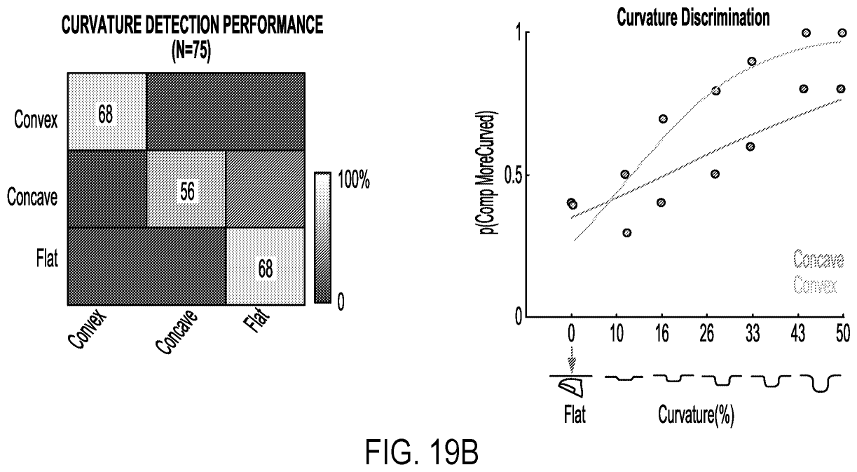

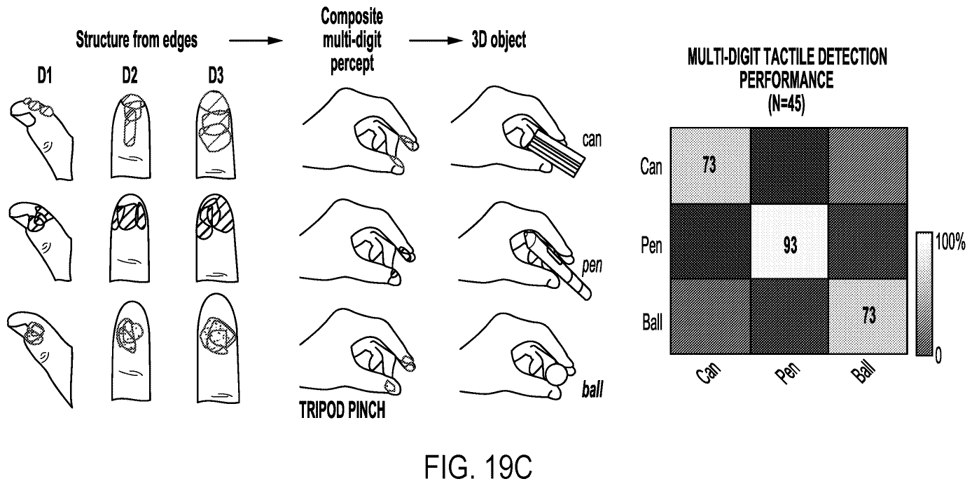

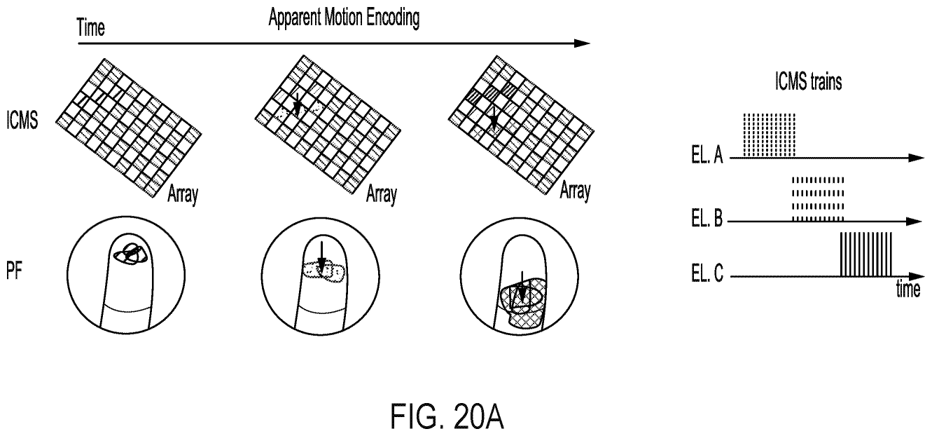

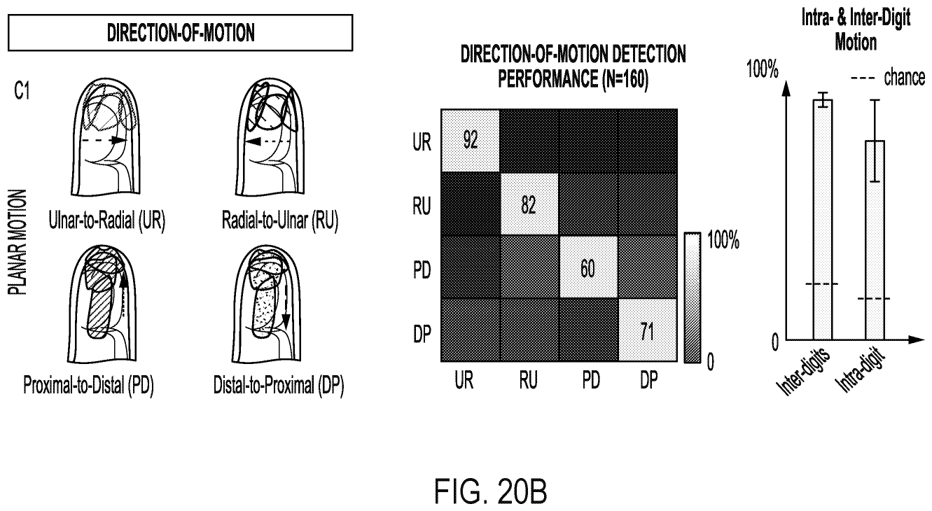

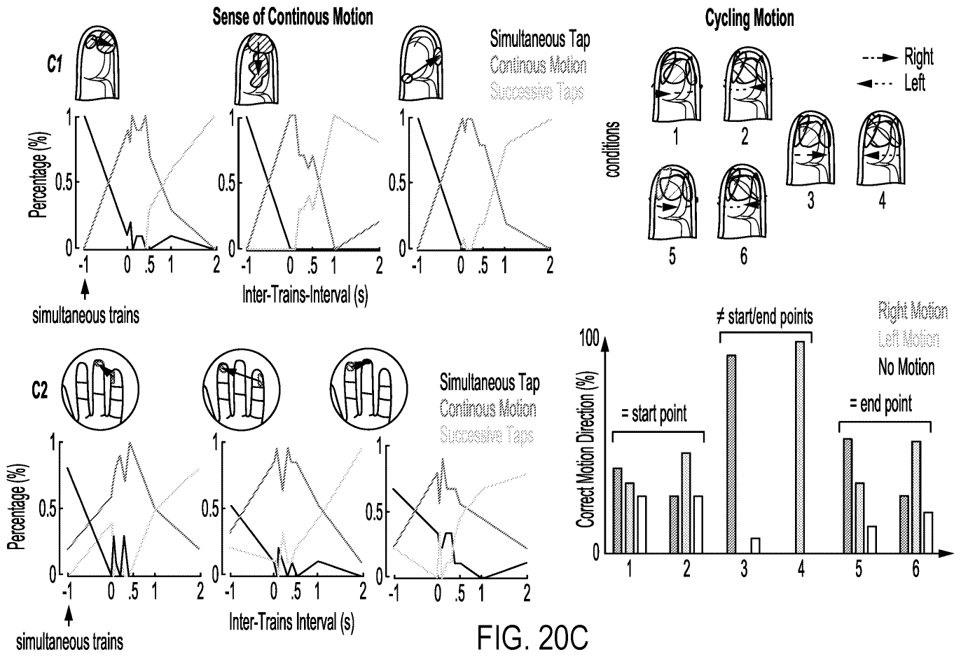

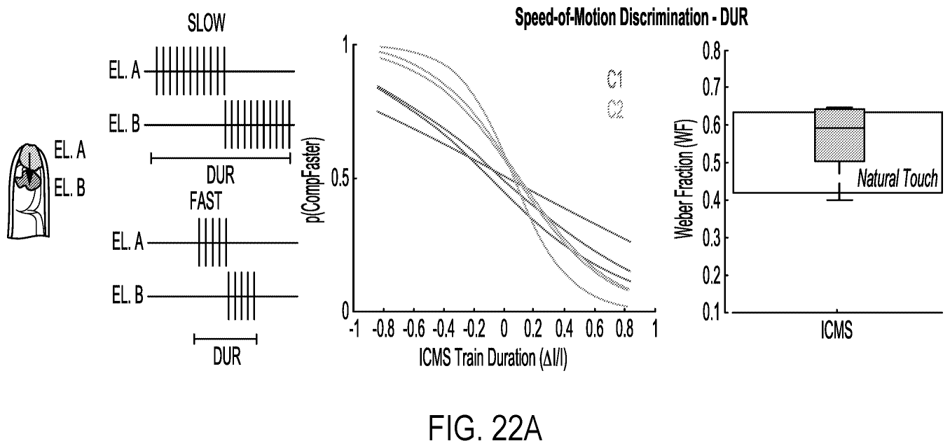

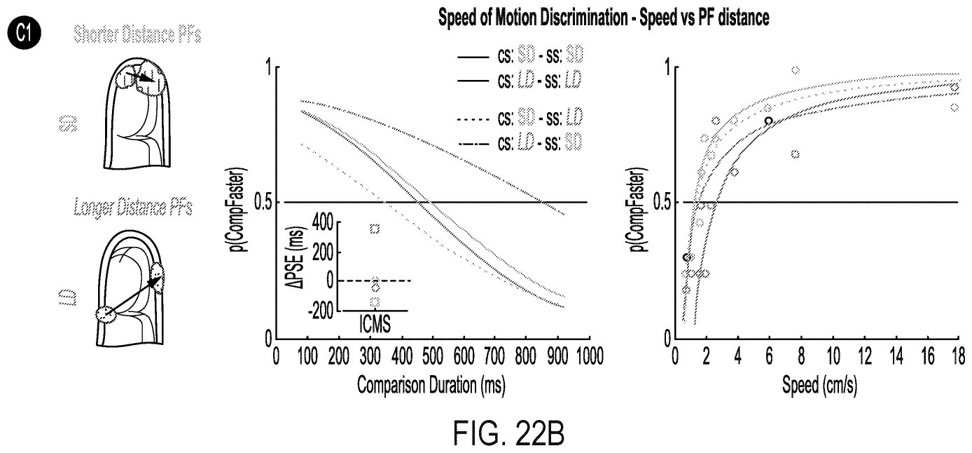

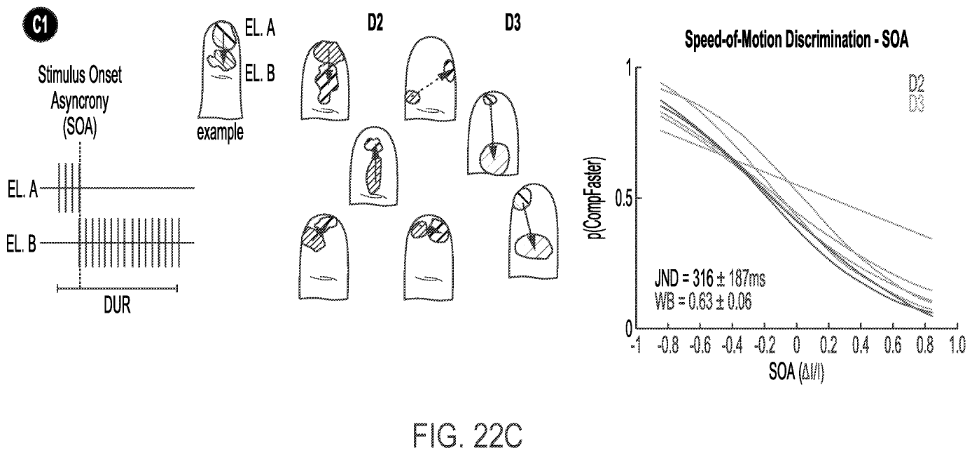

The experiments described herein include two participants with cervical spinal cord injuries having been implanted with microelectrode arrays in the hand representation of S1 (Brodmann's area 1, as depicted in FIG. 16A), localized based on pre-operative imaging. In both participants, stimulation through the S1 array resulted in distinct tactile sensations on their contralateral hands. From the survey data collected during a clinical trial, over the previous years, the area of skin over which the PF extended and its center of mass (centroid) was computed for single and multichannel stimulation. This allowed for the estimation of which combinations of electrodes can reliably evoke aligned PFs (as depicted in FIG. 16B). Then, multiple electrodes were stimulated with adjacent PFs simultaneously, which evoked perception of edges (as depicted in FIGS. 17A-17C) and arbitrary shapes (as depicted in FIGS. 18A-18D) indented into the skin. Second, it was demonstrated that the difference in the relative amplitude of stimulated electrodes lead to a perception of a curved indentation, which evoked a vivid perception of distinct object being held when stimulating multiple fingers (as depicted in FIG. 19). Third, stimulating electrodes in a temporal sequence evoked the sensation of motion across the skin at a certain speed (see FIGS. 20A-20C, 21A-21B, and 23A-23B). Lastly, combining the spatial with temporal principles of organization, even more complex shapes were evoked (see FIGS. 22A-22C).

FIG. 16A depicts four Utah arrays (Blackrock Neurotech, Inc.) implanted in participant C1 and C2, two of which were placed in the hand representation of S1 (Brodmann's area 1), based on localization with brain imaging techniques (fMRI and/or MEG). Here array locations are shown based on intra-operative photos, superimposed on a pre-surgical anatomical MRI. The central sulcus is indicated by the dashed line. M1 indicates the primary motor cortex, while S1 indicates the primary sensory cortex. Directionally, L means lateral and A means anterior.

FIG. 16B depicts locations of projected fields—the location on the hand where sensations are experienced—for each S1 channel for participant C1. The top array is medial while the bottom one is lateral. Colors denote the location of the projected field. Gray squares denote electrodes that evoked sensations on the dorsum of the hand, and white squares denote unwired electrodes. Zoomed parts show precise locations of single electrode PFs on D2 and D3 and the possible combinations tracing different tactile shapes.

### Conveying Tactile Percept Orientation Via Informed Spatial Patterning of ICMS

FIG. 17A depicts a Schematic of spatial organization of the stimulated electrodes evoking sensations of Vertical, Horizontal and No orientation (Neither) edges on the microelectrode array and their corresponding projected fields.

FIG. 17B depicts projected fields that formed horizontal (red), vertical (blue) edges, or non-specific shapes (green) on thumb (D1), index (D2), and middle (D3) fingers.

FIG. 17C depicts orientation identification accuracy for each digit. Columns correspond to the true label of the stimulated set of electrodes, and rows to sensation identified by the participant. Numbers in the boxes indicate the percent of trials this stimulus was identified out of the times it was presented, with N=150. Data sourced from participant C1.

FIG. 18A depicts examples of combinations (2 and 3) of single electrode PFs to create arbitrary tactile shapes on D2. Composite percepts reported by C1 when stimulated with the relative combination. Shaded area represents the area of sensation. Thick lines indicate the zone where the sensation is stronger/more intense. Black lines are the predictions of the evoked sensation from the single electrode PFs.

FIG. 18B depicts identification performance of five arbitrary tactile shapes, combining 2 PFs on D2 (upper chart) and D3 (lower chart), randomly presented to participant C1. Numbers in the boxes indicate the percent of success identifications per condition.

FIG. 18C depicts arbitrary shapes recognition broken down by participant (C1 and C2) and digit (D2, D3) in the upper chart, and arbitrary shapes recognition broken down by ICMS-evoked edge complexity (2 and 3 combined PFs) in the lower chart. Data for the lower chart is from participant C1.

FIG. 18D depicts a schematic of predicted and perceived ICMS-evoked sensation angle and length, as well as Pearson's Correlation between predicted and perceived sensation angles in both participants C1 and C2 in the center chart. The rightmost chart depicts Pearson's Correlation between predicted and perceived sensation lengths in both participants C1 and C2.

After establishing that ICMS through electrodes with adjacent PFs forming a line evokes discernible percept of an edge with specific orientation, the experiment described herein verified the ability to evoke more complex shapes by stimulating electrodes whose PFs form varied curves. To encode arbitrary tactile shapes over the skin, the degree to which the participants' judgment of the tactile experience matched the expected percept based on the PFs of the individual electrodes (as in FIGS. 18A-18D) was assessed.

This experiment evaluated multiple combinations of electrodes (2-PFs or 3-PFs), with PFs distributed on index and middle fingers (D2 and D3) for both C1 and C2, and the participants were asked to draw the area of sensation on the hand as well as the zone with dominant intensity (if present). The participants spontaneously reported sensations of edges and shapes when multiple electrodes with aligned PFs were simultaneously stimulated. Data was collected over 4 days within one month for each electrodes' combination.

Next, the experiment calculated average shapes corresponding to each tested combination of electrodes in both C1 and C2. Finally, the length and angle of these shapes were compared to the ones predicted by PFs of individual electrodes (as depicted in FIG. 18D, specifically lines passing through the centroids of the individual channels PFs).

Significant correlations were found for both angles and lengths (see the graphs in FIG. 18D), showing that the perceived shape corresponds to the spatial organization of PFs.

To demonstrate the discriminability of the evoked sensations, participants were asked to choose one out of three (in C2) or one out of five (in C1) shapes visually presented to them on a screen in response to multichannel ICMS. Both C1 and C2 were able to identify correct shapes (FIG. 18C, upper chart) both on D2 (C1: 82±9.2%, chance 20%; C2: 50±14%, chance 33%; Fisher Exact test, p<0.05, FIG. 18B, upper chart) and D3 (C1: 72±8%, chance=25%; C2: 48±8%, chance 33%; Fisher Exact test, p<0.05, FIG. 18B, lower chart).

Due to the rich PFs map of C1, more complex edges were tested, which combined three PFs in D2 and D3. The subject was able to correctly recognize which 3-PFs edge was presented (60±22.7% on D2, chance 25% and on 57±7.6% D3, chance 25%, Fisher Exact test, p<0.05). Notably, as in natural touch, the performance decreased with higher edge complexity (FIG. 18C, lower chart).

To further examine the encoding of even more complex shapes, the experiment also leveraged the highly detailed PF map of C1 to encode five different letter-like forms on the D2 finger pad. The experiment simultaneously delivered ICMS on multiple electrodes (from 3 to 7) asking the participant to draw the shape of the evoked percept, as in the previous task. The tests were then repeated 4 days over the span of a month. Without instruction, the participant was able to reproduce letter-like shapes that corresponded to each sequence. Results from the average drawings shows the possibility to evoked complex composite percepts (such as letter-like shapes), starting from PF of the individual electrodes.

### Spatial ICMS Modulation for Encoding Curved Tactile Surfaces

FIG. 19A depicts encoding edge curvature through spatio-temporal pattering of ICMS. Electrodes with aligned PFs were chosen to evoke a percept with a specific orientation. Then, amplitude and synchrony between the activated channels were modulated in order to encode convex or concave stimuli on D2.

FIG. 19B depicts curvature recognition performance with 3 different stimuli: convex, concave, and flat edges (left chart). N=75. Data is from participant C1. FIG. 19B also depicts the results (in the right chart) of a curvature discrimination task, in which participant C1 was asked to compare the curvature of a flat surface to other with different curvatures (from 0%-flat to 50%-most curved).

FIG. 19C depicts combinations of PFs simultaneously activated on three different digits (D1, D2 and D3). PFs relate to single channel stimulation. Conditions represent stimuli on two perpendicular axes (Horizontal and Vertical) and one condition with no orientation. Multichannel ICMS evoked sensations on all the three digit simultaneously. Participant C1 reported to perceive to grasp real objects (Can, Pen and Ball). Numbers in the boxes in the right graph indicate the percent of success identifications per condition, reflecting multidigit identification performance. N=45. Data is from participant C1.

The above experiments evoked roughly equal sensation on each segment of the skin. However, objects often have curves that push into the skin harder in some parts and lighter in the others. When the skin is deformed by a curved object, the points of pressure and the temporal activation of the mechanoreceptors depend by the specific curvature and the local shape (e.g., convex and concave).

Thus, this experiment assessed the ability to convey the sensation of a convex or concave surfaces by modulating relative strength of electrodes whose projected fields form a line. Specifically, the amplitude of stimulation of adjacent electrodes was offset by a constant value, which also led to a delayed sensation at the sites corresponding to the deep part of the surface (FIG. 19A). During the experiment, the subject reported perceiving sensations of curved edges on the skin and was able to reliably differentiate between concave, convex, and flat curves (64±6.9%, chance 33%, n=75, Fisher Exact test, p<0.05, as seen in FIG. 19B, left chart). Furthermore, the subject could differentiate between specific curves of the edge, conveyed through different offsets of stimulation (as seen in FIG. 19B, right chart).

Additionally, to verify that this local feature can be reliably encoded, the experiment included a discrimination task where the same edge (encoded via 3 PFs aligned on D2) was randomly presented but with different level of curvature (from 0% to 50%). Results showed that C1 perceived more curved edges (both convex and 236 concave) when the curvature was higher than 20% (as also seen in FIG. 19B, right chart).

To further validate the ability of the experiment to provide complex percepts to the limit, sensation of curves and oriented edges were provided to multiple digits simultaneously. C1 reported evoked sensations resembling real objects. The feeling of three edges on three digits evoked a vivid sensation of 3D structures, described as: a pen, when all the three edges encoded were along the horizontal axis on each finger; a can when along a vertical axis; and a ball, when the PFs were stimulated on multiple digits with horizontal, vertical alignment or neither (round PFs) (as seen in FIG. 19C). To test whether this evoked sensation would be reliably distinguishable, the experiment involved a multichoice task in which the 3D objects encoded via ICMS on 9-channel whose PFs fall on multiple digits were randomly presented. Participant C1 was able to successfully identify the encoded object above chance (79±11.5%, chance 33%, Fisher Exact test, p<0.05, n=45, FIG. 19C, rightmost chart). This demonstrates that complex percepts resembling real-world sensations of objects can be evoked that can be exploited in the context of bionic hands or virtual environment to enable dexterous manipulation.

### Conveying Apparent Tactile Motion Via Informed Spatiotemporal Patterning of ICMS

FIG. 20A depicts sequential stimulation through multiple (>2) electrodes having aligned PFs allow to evoke sensations of something moving on the skin.

FIG. 20B depicts combinations of PFs sequentially activated on four different directions (Ulnar-to-Radial, Radial-to-Ulnar, Proximal-to-Distal and Distal-to-Proximal). PFs relate to single channel stimulation. Numbers in the boxes in the center graph indicate the percent of successful identifications per condition with N=160. The rightmost graph of FIG. 20B is a comparison of direction-of-motion identification performance between intra- and inter-digits motion.

FIG. 20C depicts in the left-side graphs results for testing for a sense of continuous motion. Two PFs in three configurations are simultaneously (ITI=−1 s) or sequentially (ITI between 0 s and 2 s) activated. Thick Colored Lines represent sense of continuous motion (percent); Light Colored Lines represent sense of intermittent motion (taps); and Black Lines represent no motion (simultaneous tap). N=100. The right-side graphs depict direction-of-motion starting and ending in different points on D2 (cycling motion). Blue represents evoked right movements while yellow represents evoked left movements.

Perception of continuous motion of an object across the skin was enabled by smooth transition of activity between adjacent skin receptors. The experiment verified that that effect can be mimicked by stimulating electrodes with adjacent PFs in a temporal sequence. In both participants, electrodes were selected that formed two axes: proximal-distal—PD—(along the finger) and radio-ulnar—RU—(across the finger), which formed four directions of motion (PD, DP, RU, UR) (as depicted in FIG. 20B). In C1, both axes shared the central electrode (PF), forming a cross on the index finger; in C2, the radio-ulnar movements were entirely on the middle finger, and the proximal-distal movements had PFs across index, middle, and ring finger. The electrodes within a sequence forming a direction were stimulated for 500 ms each with 10 ms delay after the beginning of stimulation of the previous one.

The participants' task was to report the direction of the planar movements on the skin. Without prior experience or prompting, the participants immediately described the evoked sensation as “something moving on my fingertip” or “I am feeling like I am rolling my finger on a surface.” Results showed that both participants were able to distinguish 4 different directions of motion (C1: 76±13.8%, chance 25%, Fisher Exact test, p<0.05, see FIG. 20B; C2: 78=14.7%, chance 25%, Fisher Exact test, p<0.05, as seen in the center graph of FIG. 20B), independent of the intensity of stimulation.

The sensation of continuous motion was evoked in a specific range of delays in the onset of stimulation (as seen in the right-side graphs of FIG. 20C). If the stimuli were delivered with temporal overlap between trains (negative inter-trains interval, ITI, which may alternatively be referred to as inter-stimulus interval, ISI) the stimulations tended to be perceived as simultaneous by the users. The sensation stated to become perceived as continuous motion when ITIs spanned a range between Oms and 500 ms, otherwise they were perceived as successive, but distinct taps (intermittent motion, often with temporal gaps ≥1 s). This result was consistent regardless of the specific PF locations and separation. These thresholds match the ones observed as in natural touch.

Next, the effect of different ICMS durations and amplitudes on participant's ability to determine direction of motion was assessed. The duration of the ICMS train has a significant effect on the ability to distinguish motion direction (50 ms-30%, 200 ms-72%, chance 25%, Fisher Exact test, p<0.05). These findings are coherent with the properties of natural touch. As for orientation, this result shows that the participants would need only tens of milliseconds to successfully interpret this local tactile feature, practical for use in a closed-loop BCI.

The effects of amplitude on the perception of tactile motion were then tested, delivering the stimulus to the set of 3 electrodes at either 40, 60, or 80 μA. The participant was able to distinguish direction of motion at each amplitude (40 μA-53%, 60 μA-71%, 80 μA-66%, chance 25%, Fisher Exact test, p>0.05, n=270), showing minimal effect of intensity on the performance. In other words, even though the intensity of the perceived sensation is modulated by stimulation amplitude the local property of tactile motion remains distinguishable by the user.

To assess whether the participants were using information about terminators (or the starting position) to judge motion direction, stimulation was delivered (encoding left or right directions) cycling through the electrodes (three times on each trial) but starting from a random electrode within the set (FIG. 20C, right-side graphs). The findings showed that the participant relied on both the starting and the terminating channels (i.e., terminators) to judge motion, and it appears to be that PFs which are contiguous on the skin evoke a smoother sensation of apparent motion.

Furthermore, the same strategy of dynamic ICMS pattering, delivered through electrodes with aligned PFs, was adopted for encoding other types of apparent tactile motion, such as circular and radial motion. In the first case, three combinations of three electrodes with PFs aligned in a circular fashion were sequentially stimulated in a clockwise, counterclockwise or rectilinear direction. C1 had to distinguish which direction of motion was presented. In the second experiment, radial motion was encoded using a single channel as a center point (small PFs) and a set of three radial channels (large PFs) whose PF centroids were roughly equidistant from the central single channel.

In the trials encoding motion contraction, the three radial channels were initially activated simultaneously and then the single central channel. The opposite order was adopted to encode motion expansion. For static condition, the single central electrode was activated sequentially twice. From the performances of the participants, the dynamic activation of multiple electrodes can encode, not only planar, but also apparent circular (96±7.5%, chance 33%, Fisher Exact test, p<0.05) and radial motions (81±16.6%, chance 33%, Fisher Exact test, p<0.05).

### ICMS-Evoked Motion Percepts Across Digits

After assessing the intra-digit apparent motion, the participants were asked to judge direction of motion across digits. Electrodes were selected evoking PFs on D1-D2-D3-D4 for C1 and D2-D3-D4 for C2. Then, stimulation patterns were randomly presented for two different motion directions across digits, or simultaneously activated all the channels (no motion). Both subjects were able to detect motion as well as the correct direction (C1: 98±2.5%, n=120; C2: 75±14%, n=75, Fisher Exact test, p<0.05). Relative to the intra-digit motion tasks, C1's performance was statistically higher for the inter-digit motion discrimination tasks (+22%, Fisher Exact test, p<0.05) (as seen in the rightmost graph of FIG. 20B). A notable application of apparent inter-digit motion in the context of bionics hand is the encoding of real-time object slippage, particularly important during object manipulation.

### Encoding Motion Through Amplitude Modulated ICMS

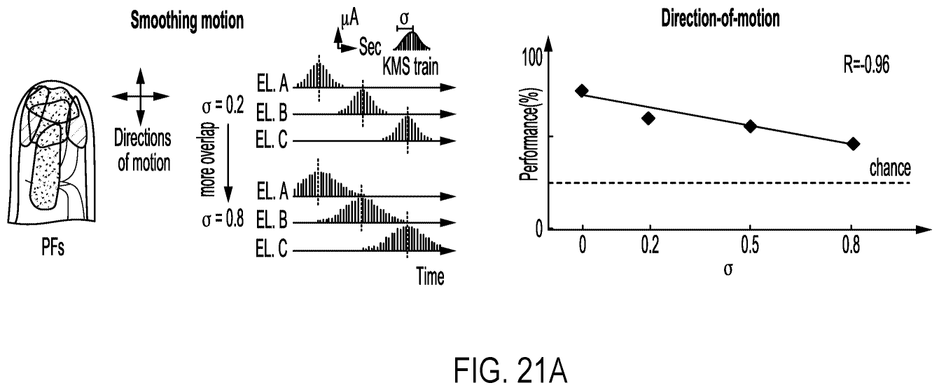

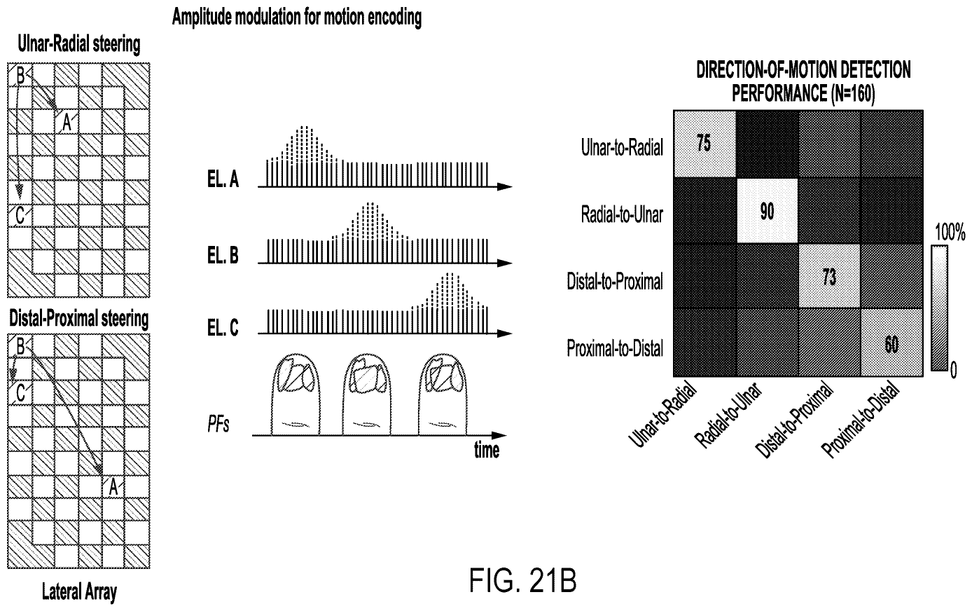

FIG. 21A depicts four directions of motion on D2 that were encoded through multichannel ICMS. Smoothing motion between PFs has been defined as overlapping gaussian-shaped trains on different channels (varying sigma—σ—from 0 to 0.8). FIG. 21A also depicts in the right-side graph performance according to the overlap between the ICMS trains on different channels.

FIG. 21B depicts the modulation of amplitude among multiple channels, allowing for shaping the applied voltage. FIG. 21B also depicts in the right-side graph direction-of-motion recognition performance adopting amplitude modulation. Three PFs are involved in the encoded movements. Numbers in the boxes indicate the percent of success identifications per condition.

As a person explores an object or texture through touch, they feel local features like small bumps or dips traveling on the skin at the same time as they maintain the sensation of overall contact with the object. To investigate the possibility of using more efficient dynamics of ICMS than fixed-amplitude trains like those proposed for intracortical visual prostheses (i.e., dynamic current steering—DCS—or voltage field shaping) and cochlear implants, the embodiments herein contemplate patterns of ICMS stimulation whose amplitudes are variably modulated individually on each channel in order to elicit the perception of apparent motion (as seen in the left-side graph of FIG. 21A). This was then repeated the direction-of-motion task using dynamic ICMS train (amplitude between 40 and 80 μA modulated through a Gaussian-shape dynamic between multiple electrodes). Discrimination performance was above chance and similar to the one achieved with the sequential activation of the same channels (without DCS: 76% and with DCS: 74%, chance 25%, Fisher Exact test, p<0.05). Furthermore, the same discrimination task was performed again, changing the level of overlap between the ICMS trains (σ of the Gaussians ranging from 0.2 to 0.8) on different sequentially activated electrodes. The performance decreased as the overlap between trains increased (r=−0.96, p<0.001, as seen in the right-side graph of FIG. 21A), showing a negative effect of smoothing the spatio-temporally modulated ICMS trains on apparent tactile motion discrimination.

Also assessed was whether the amplitude modulation could be adopted to encode motion as an additional tactile feature in conjunction with contact events which necessarily precede the sensation of motion. When a person grasps an object, neurons with the receptive field on the contact area start to fire, then when that same object starts to move over the skin in a certain direction, the person's sensory system is able to encode that motion via temporal modulation of the firing. To artificially encode this scenario, the experiment simultaneously stimulated three electrodes with aligned PFs and shaped the voltage fields across these three electrodes (as seen in the left-side graph of FIG. 21B). Also with voltage field shaping, the participant was able to identify motion among 4 directions on the skin (D2) (C1: 74±12.3%, n=160, Fisher Exact test, p<0.05, as seen in the right-side graph of FIG. 21B). This result showed the ability of the subject to simultaneously disentangle three distinct tactile features from ICMS: location, intensity, and motion direction of the artificial percept.

### Patterns of Multichannel ICMS can Modulate Motion Speed.

The experiment also investigated how spatiotemporal parameters of stimuli can affect the speed of tactual apparent movement. Perception of motion speed is useful in artificial touch for encoding important object interactions, such as slippages or surface exploration. The purpose, then, was to validate how stimulus properties can be mapped onto percepts of tactual apparent movement with varying speeds.

The experiment adopted two different encoding schemes based on the modulation of ICMS train duration and stimulus-onset asynchrony (SOA) as depicted in FIGS. 22A-22C. In the first strategy, the ICMS total duration was modulated, making the change between 2 PFs quicker or slower. The second encoding strategy modulated the TC (time of change or stimulus onset) between the two stimulating channels (and so the two PFs), keeping constant the total train duration (2 sec), aiming to evoke a faster apparent motion for lower TC. In a speed discrimination two-alternative forced choice (2-AFC) task, the participants were asked to identify which of the two sequentially presented ICMS patterns they perceived as faster (as seen in FIG. 22A). Results from all tested electrodes' pairs showed that both subjects were able to discriminate speed of motion via ICMS train duration (Weber fraction=0.56±0.09) which falls in the same range as natural touch. Both participants described the modulations as changes in speed of motion.

To ensure that the participant was identifying speed of motion rather than train duration alone, the same task was repeated, presenting standard and comparison stimuli on two different distances keeping the same ICMS train durations as seen in FIG. 22B. Channels with short (SD, inter-centroids distance of ~0.6 cm) and long distances (LD, inter-centroids distance ~1.4 cm) were selected on the same digit (D2) and used both as standard stimuli. The PSE (point-of-subjective equality) shifted according to the motion distance, confirming the hypothesis of perceived speed (Δdistance/ICMSduration−cm/s). Indeed, when both standard (500 ms) and comparison (from 79 to 921 ms) stimuli were presented on the same distance (SD and LD), the APSEs were approximately Oms. On the contrary, when the standard stimuli were provided on SD and compared to stimuli on LD, and vice-versa, the APSE was +360 ms (PSE speed=1.63 cm/s) and −140 ms (PSE speed=1.67 cm/s) respectively.

Additionally, ICMS trains with different stimulus-onset asynchronies (SOAs) were interpreted as modulating the speed of motion both on D2 and D3 in C1 (JND=316±187 ms, Weber fraction=0.63±0.06, as seen in FIG. 22C). In addition, different encoding strategies modulating the inter-stimulus interval (ISI) or temporal separation and the temporal overlap between the trains (TO) were tested on different electrodes. Both these encoding strategies failed to modulate speed of the apparent tactile motion.

These findings demonstrate that the frequency of perceived movement is greatest when a temporal overlap is fixed (of approximately 100 ms) without modulation, and other variables simply are set.

### Sequential ICMS Improves Perception of Complex Tactile Shapes

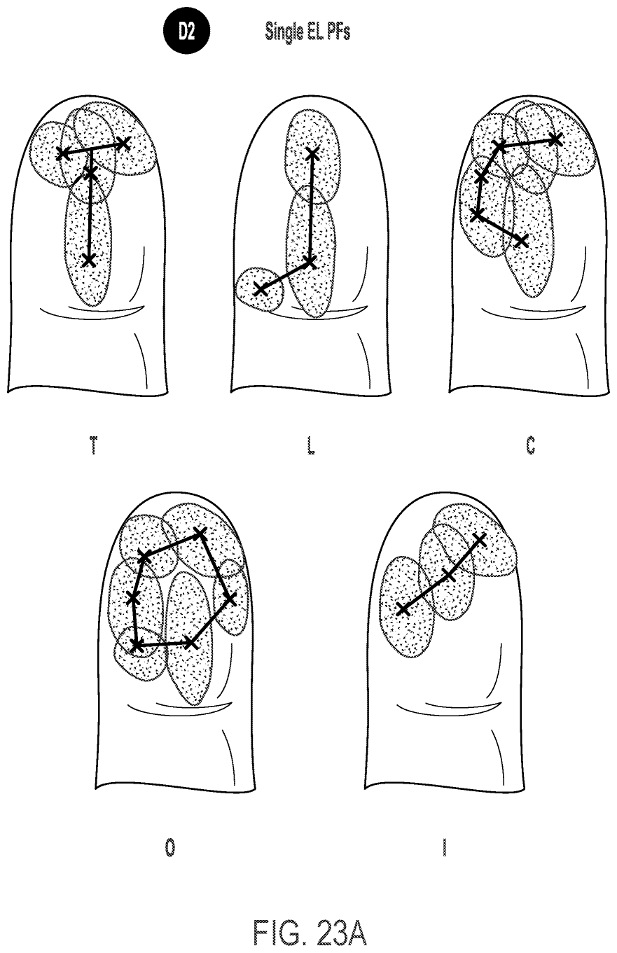

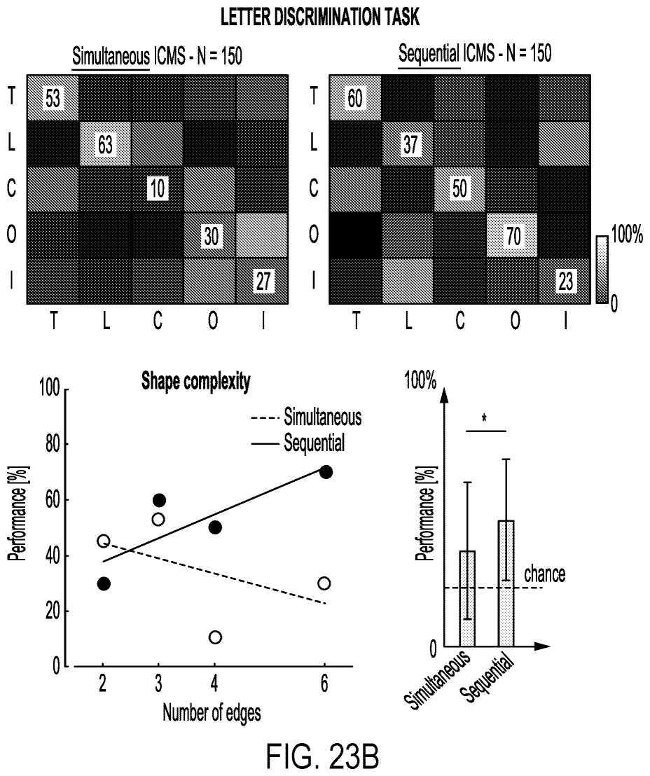

FIG. 23A depicts a selection of PFs (>3) creating shapes of letter on D2.

FIG. 23B depicts the results of a letter recognition task encoded using sequential or simultaneous ICMS. Numbers in the boxes in the upper graph indicate the percent of success identifications per condition, with N=150. FIG. 23B also depicts identification performance according to shape complexity (lower left) and direct performance comparison between sequential and simultaneous ICMS (lower right), with *p<0.05.

### Combinations of Aligned ICMS-Evoked PFs Result in Composite Tactile Sensations

Multichannel ICMS as described herein can be used to improve the properties of artificial touch. The advantage of multi-channel ICMS is that it gives rise to a wider dynamic range of sensations, which can thus convey more forces. However, selecting specific channels with distributed PFs to evoke tactile edge sensations and custom shapes, has not been previously demonstrated. The foregoing disclosure demonstrates that the use of specific spatio-temporal patterning of ICMS as described herein can evoke composite tactile percepts by combining electrodes with aligned PFs. These individual ICMS-evoked PFs are analogous to tactile ‘qualia’ and can be combined to generate coherent tactile forms and motion.

Electrodes are located at discrete locations on the cortex (BA1), allowing specific combinations to stimulate cortex in a continuous trajectory. In the context of natural touch, somatosensory cortex processes natural touch through a specific hierarchy: neurons in the earliest stages of sensory processing—area 3b—respond to the motion and orientation of local elements, whereas neurons in higher processing stages—area 1, where the electrodes are implanted—are apt to encode global motion and shapes, for example, when stimulated with random dot patterns.

Voltage field shaping can be used to encode apparent motion in artificial touch. In this specific case, the participant reported to perceive “an edge moving over the skin” in a specific direction.

Both encodings of tactile edge orientation and motion via informed spatial patterning of ICMS were achieved regardless of the specific properties of the individual PFs, apart from their relative location on the homunculus. Indeed, the parameters of ICMS (amplitude, pulse-width, and frequency) were the same for each stimulating channel (except for encoding curvature). Composite sensations of shapes and apparent motion have been evoked consideration of other PF qualities, geometry, size, or perceived intensity.

In addition, multichannel PFs are perceived as a composite sensation, without spatial gaps, when provided on the fingertip with a proximity in the same range as in natural touch (e.g. two-point discrimination thresholds between 2 to 8 mm on fingertips). This finding is line with theories based on the funneling illusion, suggesting that cortical activation corresponds to the perceived, rather than actual, site of peripheral stimulation and that spatial perceptions are strongly dictated by central representations. Indeed, this stimulation of multiple electrodes can lead to a spatial integration and a single cortical activation zone.

In addition, participants were able to recognize several tactile shapes encoded through multichannel ICMS. Notably, they made more confusions in recognizing the correct tactile shape when the combinations shared similar PFs location or combination of electrodes adopted in the multichannel set. For example, on D3, C1 often confused E2 and E3 both having a common electrode in the set and the resulted PF was on the medial side of the fingertip for both. Same for 3-PFs combinations, where E1 and E2 on D3 resulted in very similar PF.

Just as spatial sensitivity matches natural touch, ICMS train duration influenced the ability to detect both edge orientation and apparent motion, in a manner similar to its natural counterpart. ICMS duration was used to modulate speed of motion, obtaining in both participants discriminability comparable to that of natural touch. Notably, sensations of continuous motion were reported when the ICMS trains were delivered with ITIs lower than 500 ms (as seen in FIG. 20C). Higher ITIs generated percepts described as two successive skin taps, rather than motion. This is consistent both with natural touch and vision. Indeed, the perception of apparent movement depends on the time interval between sequential taps or flashing lights.

Edge Curvature and Three-Dimensional Object Shape from Artificial Touch.

Mechanoreceptors in the glabrous skin are sensitive to the edges of stimuli, suggesting that the effective stimulus for these receptors is the curvature of the skin. The skin deformation (and the points of pressure) changes according to the level of curvature and the contact area. The human tactile discrimination of curvature is around 10% both for concave and convex objects and depends on the contact areas between the curved surfaces and the finger pad skin. With spatiotemporally modulated ICMS, the participant perceived curved shapes indenting the skin and was able to discriminate curvature above 20%.

The sense of touch is of major importance for the perception of three-dimensional shapes. In actively dealing with objects, both the cutaneous sense (input from receptors in the skin) and the kinesthetic sense (input from receptors located in muscles, tendons, and joints) convey information. Object perception, originating from such combined inputs, is termed haptic perception or stereognosis.

The three-dimensional shape encoding that leverages tactile local features on multiple digits has not been previously explored. In the absence of vision, object perception results mostly from the integration of specific tactile information including contact points, curvatures, texture and local edges. The multi-digit cutaneous inputs are integrated in cortex to form central representations of objects that are matched against memory and perception. This shows that specific oriented edges (local tactile features) evoked on multiple fingers result in vivid three-dimensional shapes associated with objects of everyday life (e.g., pen or spheres). When coupled with visual feedback (e.g., a robotic hand grasping physical objects), these artificial sensations may result in even more natural percepts due to the additionally provided context.

### Tracing ICMS Trajectories on the Cortex Allows for Encoding Complex Tactile Shapes.

Looking at natural tactile perception, it is possible to recognize numerals or letters when they are drawn in the skin (i.e., graphesthesia). For instance, active touch has been shown to be better than static deformation of the skin for discriminating shapes. The experiments described herein activated electrodes, with adjacent PFs on the skin, encoding tactile letters through sequential or simultaneous ICMS. Without any training, C1 was able to reliably identify, name, and discriminate these forms. Tracing trajectories on the somatosensory cortex with sequential ICMS allowed for a better understanding of the traced shapes, regardless the shape complexity.

While this experiment tested only letter-like shapes, the outlines of other common objects, this could also be traced using the same principles. These results favor the reduction of current injected into the cortical tissue at a single given point, reducing the likelihood of epileptic seizures due to synchronized activation of multiple electrodes. When multiple electrodes are simultaneously stimulated, the resulting PF can interact, often coalescing into a single PF that is not always described as a shape. Dynamic stimulation diminishes the likelihood of this issue, improving the effective spatial resolution of the patterns that can be evoked.

Direct electrical stimulation activates all neurons in the proximity of the electrode, regardless of their feature selectivity, an unnatural activity affecting sensory perception, both in the context of visual and somatosensory neuroprostheses. The findings herein suggest the possibility to restore complex tactile percepts via ICMS of S1 in the context of bionic hands.

### Cortical Spatial Arrangement of the Electrodes.

Considering the electrode configurations adopted in the embodiments herein, it is possible to depict the cortical arrangements, based on cortical somatotopy, for obtaining specific PFs. This disclosure that the perceived edge length correlated significantly with the degree of separation (i.e., cortical distance) between electrodes. This suggests that the edge length and appearance is not only a function of the number of electrodes being stimulated, but also of their spatial distribution in the array.

Along the same line, the directions of motion reported by the subjects are tracing specific cortical trajectories in the brain. Indeed, when apparent motions are crossing the skin, the cortical trajectories are also crossing cortex in the brain. The results herein suggest that more electrodes can be used to cover the whole hand area, and a higher stimulation selectivity could allow for an even more detailed map of PFs. These high-resolution maps could restore the same acuity and richness as natural touch. Furthermore, high-density penetrating electrodes could activate a smaller volume of cortex, reducing the likelihood of interactions between adjacent electrodes in both static and dynamic stimulation paradigms.

### Methods

Participant C1 (male), 57 years old at the time of implant, presented with a C4-level ASIA D spinal cord injury (SCI) that occurred 35 years prior to implant. Participant C2 (male), 60 years old at the time of implant, presented with C4-level ASIA D spinal cord injury (SCI) and right brachial plexus injury that occurred 4 years prior to implant.

### Cortical Implants

The experiments herein implanted four microelectrode arrays (Blackrock Neurotech, Salt Lake City, UT, USA) in each participant. The two arrays (one medial and one lateral array) in Brodmann's area 1 of somatosensory cortex were 2.4 mm×4 mm, with sixty 1.5-mm long electrode shanks wired in a checkerboard pattern such that ICMS could be delivered through 32 electrodes. The two arrays in primary motor cortex were 4 mm×4 mm, with one-hundred 1.5-mm long electrode shanks wired such that 96 electrodes could measure neural activity. The inactive shanks were located at the corners of these arrays. Two percutaneous connectors, each connected to one sensory array and one motor array, were fixed to the participant's skull. We targeted array placement based on functional neuroimaging (fMRI) of the participants attempting to make movements of the hand and arm, within the constraints of anatomical features such as blood vessels and cortical topography.

### Intracortical Microstimulation (ICMS)

Stimulation was delivered via a CereStim 96 (Blackrock Neurotech). Each stimulating pulse consisted of a 200-μs cathodic phase followed by a half-amplitude 400-μs anodic phase (to maintain charge balance), the two phases separated by 100 μs.

### Simultaneous and Sequential Multi-Channel ICMS

The experiment herein selected groups of 2/7 electrodes. In most cases, the electrodes had aligned projected fields (the patch of skin over which the ICMS-evoked sensation is experienced). For C1, each of the selected electrodes evoked a PF in a single digit (D1, D2 or D3). For C2, the selected electrodes were organized in groups (from 2 to 5) evoking then a PF in a single (D3) or multiple digits (D2-D3 or D3-D4). When stimulating through multiple electrodes, all electrodes delivered the same ICMS pulse train simultaneously for encoding edges. Contrary, for motion encoding the electrodes were activated, with the same ICMS parameters, in a specific sequential order (with different ITIs). During each experimental block, stimulation through each electrodes' combination was randomly interleaved.

### Projected Fields

Projected fields were collected over multiple years for C1 or months for C2 for each electrode or electrode groups. On each trial, a 60 μA, 100-Hz ICMS train was delivered through a given electrode or group and the participant drew the spatial extent of the sensation on a digital representation of participant hand. The participant could request as many repetitions of the stimulus as desired. The region enclosed by the drawn boundary constituted an estimate of the projected field for that electrode on that session. PFs obtained for each electrode or group were combined across sessions to obtain a time-averaged estimate (such as that shown in FIG. 16B). From these digital images, the PF centroid (center of mass) was also computed. An aggregate PF was then computed for each stimulating channel by weighting each pixel on the hand by the proportion of times it was included in the reported PF over the duration of the study. This allows for an estimate of which combinations of electrodes would reliably evoke aligned PFs.

### Edge Orientation Task

The Edge Orientation Task was designed to assess participants' ability to identify the orientation of tactile sensations induced by simultaneous multi-channel ICMS. In this task, participants were presented with tactile sensations through multiple electrodes, each aligned along different axes (vertical or horizontal on the fingertip or neither). They were then asked to report the orientation of these sensations. Participants performed this discrimination task in a forced choice discrimination paradigm. On each trial, a stimulus lasting 2 seconds was presented and the participant reported stimulus orientation. The order of presentation of the stimuli was randomized and counterbalanced. Data were obtained from each electrode combination over a minimum of 5 experimental blocks, each consisting of 10 presentations of each stimulus. The frequency of the ICMS stimuli was 100 Hz. This task was performed by C1 on D1, D2 and D3. The accuracy of their responses was evaluated by comparing them with the actual orientation of the ICMS-induced percepts. This approach aims to understand the perceptual resolution and orientation discrimination capabilities of participants experiencing ICMS-induced percepts. The task was repeated varying both amplitudes (40, 60, 80 μA) and duration (250 ms, 500 ms, 1 s, 2 s, 3 s) for each stimulus orientation. The stimulation parameters were the same on each electrode.

### Multi-Channel PFs Drawings

The Multi-channel Projected Fields (PFs) Drawing task aimed to explore how participants perceive and represent tactile percepts induced by multi-channel ICMS. After experiencing these sensations, participants were asked to draw the perceived patterns on a digital hand representation. These drawings were then analyzed to assess their correlation with the expected tactile percepts, based on the stimulated electrode positions. This task provided insights into how multi-channel ICMS can be used to convey complex tactile information. Single electrodes were chosen to evoke PFs on the individual digit having the lowest detection thresholds for that specific patch of skin. The estimated shapes from multichannel ICMS are calculated based on the centroid locations of the correspondent individual PFs. A line passing through all centroids represented the estimated tactile shape. At the beginning of each session, each PF was double checked to confirm its local characteristics. Tactile shape complexity was varied from low complexity (hotspots from a single PF) to intermediate complexity (lines and curves from 2 or 3 PFs), to “highly complex” percepts (letters from >3 PFs). To compare the ICMS-evoked edges with those estimated from the adopted electrodes, their predicted and perceived angles and lengths were calculated (Pearson's correlation, FIG. 18D).

### Arbitrary Shapes Classification

The Arbitrary Shapes Classification task, building off of the Multi-channel PFs Drawing task, investigates participants' ability to classify various shapes encoded through combinations of different electrodes in multi-channel ICMS. Different shapes were represented by specific electrode combinations, and after experiencing the induced sensations, participants were asked to classify these shapes from a list of visually displayed options. The accuracy of their classification was measured, providing valuable data on the effectiveness of shape representation through ICMS. The tasks were performed both on D2 and D3. Participants performed this discrimination task in a forced choice discrimination paradigm. On each trial, a stimulus lasting 2 sec was presented and the participant reported stimulus shape. The order of presentation of the stimuli was randomized and counterbalanced. Data were obtained from each electrode combination over a minimum of 4 experimental blocks, each consisting of 10 presentations of each stimulus. The frequency of the ICMS stimuli was 100 Hz and the amplitude 80 μA. The stimulation parameters were the same on each electrode.

### Curvature Discrimination and Classification Tasks

For encoding tactile curvature, trapezoidal ICMS traces (2-sec in duration, 0.2-sec ramps) were delivered with a time offset on 3 electrodes having aligned PFs. On the electrode evoking PFs in between the other two electrodes, the ICMS trains started earlier and lasted more (0.2-sec). Moreover, its peak amplitude was also set higher (depending by the curvature level-%). This was implemented to emulate the interaction of the finger pad with a curved object. Indeed, the receptive field activations would be not simultaneous, and the force applied on the skin would be centered on the curve peak.

C1 performed a curvature discrimination task in a two-alternative forced choice paradigm. On each trial, a pair of stimuli, each lasting 2 sec, was presented with a 2-sec inter-stimulus interval and the participant reported which stimulus was more curved. The standard stimulus, consistent across the experimental block, was paired with a comparison stimulus whose curvature (amplitude and time occurrence of the central channel) varied from trial to trial and spanned the standard curvature (0% to 50%). The order of presentation of the standard and comparison stimuli was randomized and counterbalanced. Data were obtained over a minimum of 8 experimental blocks, each consisting of 2 presentations (one for each order) of each stimulus pair. The frequency of the ICMS stimuli was 100 Hz.

To test whether ICMS curvature discrimination was subject to concavity, convex and concave stimuli were selected. The former was encoded with the central channel activated earlier and set to higher amplitude while the latter adopting an opposite configuration. Curvature % represent the difference in amplitude and time onset between the adopted channels. Tests for both concavities were interleaved and randomized within an experimental block.

Then, for curvature recognition, C1 performed this task in a forced choice discrimination paradigm. On each trial, a stimulus lasting 2 sec was presented and the participant reported stimulus concavity (Flat, Convex and Concave). The order of presentation of the stimuli was randomized and counterbalanced. Data were obtained over a minimum of 5 experimental blocks, each consisting of 5 presentations of each stimulus. The frequency of the ICMS stimuli was 100 Hz.

### Object Discrimination Task

Multi-digit stimulation was provided to C1 combining the oriented edges presented in the Edge Orientation Task. Combinations of 9 electrodes (3 per digit) were adopted to encode different object shapes. Horizontal edges on three digits encoded a pen, vertical a can and otherwise a ball.

For 3D object recognition, C1 performed this task in a forced choice discrimination paradigm. On each trial, a stimulus lasting 2 sec was presented and the participant reported stimulus type (Can, Pen and Ball). The order of presentation of the stimuli was randomized and counterbalanced. Data were obtained over a minimum of 3 experimental blocks, each consisting of 5 presentations of each stimulus. The frequency of the ICMS stimuli was 100 Hz and the amplitude to 60 μA on each channel.

### Direction of Motion Tasks

The Direction of Motion Tasks evaluated participants' perception of the direction of motion from tactile percepts induced by sequential multi-channel ICMS. A single sequence of ICMS pulses across multiple (3) electrodes occurring in one of four directions (Ulnar-to-Radial, Radial-to-Ulnar, Proximal-to-Distal and Distal-to-Proximal) were used to simulate motion, and participants were tasked with reporting the perceived direction. Trains of 500 ms at 60 μA were sequentially delivered through aligned electrodes according to the specific direction. Participants performed this task in a forced choice discrimination paradigm. On each trial, a stimulus lasting 1.5 sec was presented and the participant reported stimulus direction. The order of presentation of the stimuli was randomized and counterbalanced. Data were obtained over a minimum of 3 experimental blocks, each consisting of 5 presentations of each stimulus. The frequency of the ICMS stimuli was 100 Hz. The task was repeated varying both amplitudes (40, 60, 80 μA) and duration (50 ms, 200 ms, 400 ms, 600 ms, 800 ms) for each stimulus direction. The stimulation parameters were the same on each electrode.

The same behavioral task was adopted for multidigit motion recognition, circular motion, and radial motion. In the multidigit task, participants were asked to identify the direction of motion across fingers. Same parameters were adopted for this task, while the adopted electrodes had PFs on different digits (D1, D2, D3, D4 for C1 and D2, D3, D4 for C2). In the radial motion, the adopted channels evoked PFs oriented in a circular manner on D2. Contrary for radial motion, the stimulation was provided sequentially from one channel and then from three channels for encoding contraction, while vice-versa for expansion. These specific motions were tested only in C1.

### Smoothing Motion

L Aiming to smoothing the perception of apparent motion, sequential ICMS trains between electrodes have been temporally overlapped (as seen in FIG. 21A). In detail, each ICMS trains was designed to follow an amplitude envelope as a normal probability density function:

$$y = f ( x \vert μ , σ ) = \frac{1}{σ \sqrt{2 π}} {e}^{\frac{- {( x - μ )}^{2}}{2 {σ}^{2}}}$$

The Direction of Motion Task was repeated for different levels of overlap between ICMS trains varying σ from 0 to 0.8. Overall performance was calculated as the mean recognition performance for all four directions of apparent motion. Data were obtained over a minimum of 5 experimental blocks, each consisting of 8 presentations of each stimulus. The frequency of the ICMS stimuli was 100 Hz and amplitude modulated between 40 and 80 μA following a normal probability density function. Data was collected in C1 using six PFs aligned horizontally and vertically on D2.

### Sense of Continuous Motion

The sense of continuous motion was assessed using a multichoice paradigm. Participants were asked to judge whether a stimulus was encoding a simultaneous tap, a continuous motion or two successive taps on the skin. To test this hypothesis, two PFs have been chosen and activated with different Inter-Train Intervals (ITIs). Since each train lasted 1 s, −1 s represent simultaneous delivering of the ICMS trains. ITIs of 0 s, 0.05 s, 0.1 s, 0.2 s, 0.3 s, 0.4 s, 0.5 s, 1 s, and 2 s were tested. The order of presentation of the stimuli was randomized and counterbalanced. Data were obtained over a minimum of 1 experimental block, each consisting of 10 presentations of each stimulus. The frequency of the ICMS stimuli was 100 Hz and amplitude at 60 μA. Some results from this task are illustrated in FIG. 20C.

### Motion Cycling Task

The Motion Cycling Task evaluates participants' ability to perceive and identify the direction of motion from repeated sequences of ICMS pulses across various electrodes. Unlike the Direction of Motion Task, which presents the sequence once, the Motion Cycling Task involves repeated sequences (three cycles in row) where the sequential ICMS started and ended on different PFs. Three aligned PFs on D2 were selected for this task. Each condition varies the sequence in which electrodes are activated, altering the perceived direction of motion. C1 must then report the perceived direction in sequence (Left or Right direction on the finger pad). This task sheds light on how start and end points affect motion perception accuracy and the brain's capacity to interpret motion directions according to different initiators and terminators. This task consisted in a three-alternative forced choice paradigm. On each trial, a stimulus composed by the sequential activation of three electrodes, each lasting 500 ms, was presented and the participant reported the direction of motion on the finger pad (Right, Left motion or No motion). The order of presentation of the stimuli was randomized and counterbalanced. Data were obtained over a minimum of 5 experimental blocks, each consisting of 8 presentations of each stimulus. The frequency of the ICMS stimuli was 100 Hz and amplitude 80 μA. Some results from this task are illustrated in FIG. 20C.

### Dynamic Current Steering for Motion Encoding

Dynamic current steering (DCS or voltage field shaping) was used to create virtual electrodes in between the physical electrodes on the array by using current steering. Current steering consisted of simultaneous stimulation of two electrodes in the sequence at a particular current ratio. The ratio is adjusted to change the location of the virtual electrode. The amplitude ratio varied for each individual pulse within the pulse train following a normal probability density function (see “Smoothing Motion”). The rate of change of the current ratio determines how rapidly the pattern is “drawn” on the cortex and how dynamic the pattern is perceived to be.

In detail, three channels having aligned PFs and detection threshold below 40 μA were chosen for testing DCS. All three electrodes were activated using ICMS trains having a constant baseline at 40 μA for the entire length of the stimulus (1.5 s). In addition to this stimulation, the amplitude was sequentially modulated on each channel following a normal probability density function with σ=0 for 500 ms. In this way, the voltage filed was shaped towards the next channel evoking a perception of apparent motion.

The Direction of Motion Task was repeated using DCS. Data were obtained over 5 experimental blocks, each consisting of 8 presentations of each stimulus. The frequency of the ICMS stimuli was 100 Hz and amplitude modulated between 40 and 90 μA following a normal probability density function. Data was collected in C1 using six PFs aligned horizontally and vertically on D2.

### Speed Discrimination Task

The Speed Discrimination Task tested participants' ability to discern the speed of motion from different sequences of ICMS pulses. Two sequences with varying pulse durations are delivered across two specific electrodes (or groups of electrodes), simulating different movement speeds. Participants are asked to discern which sequence represents a faster motion on skin. Participants performed a speed discrimination task in a two-alternative forced choice paradigm. On each trial, a pair of stimuli, each lasting 1 sec, was presented with a 1-sec inter-stimulus interval and the participant reported which stimulus was faster. The standard stimulus, consistent across the experimental block, was paired with a comparison stimulus whose speed (ICMS train duration or stimulus onset) varied from trial to trial and spanned the standard speed. The order of presentation of the standard and comparison stimuli was randomized and counterbalanced. Data were obtained from each electrode over a minimum of 8 experimental blocks, each consisting of 2 presentations (one for each order) of each stimulus pair. The frequency of the ICMS stimuli was 100 Hz and amplitude 60 μA. For ICMS duration, Weber fractions were calculated and reported since for C1 and C2 different duration ranges were used (stimulus standard was 500 ms for C1 and 1 s for C2). This task evaluates how well individuals can process and compare temporal variations in sensory stimuli, which is fundamental for interpreting dynamic sensations.

To test whether speed discrimination was achieved considering only ICMS train durations, two standards were selected-either motion using PFs at Short Distance (SD, inter-centroids distance of ~0.6 cm) and Long Distance (LD, inter-centroids distance ~1.4 cm)—and paired them with comparison amplitudes that spanned a range of ±500 ms around the standard. Tests for both standards were interleaved and randomized within an experimental block in C1. Data were obtained from each combination over a minimum of 8 experimental blocks, each consisting of 2 presentations (one for each order) of each stimulus pair. The frequency of the ICMS stimuli was 100 Hz and amplitude 80 μA. The conversion in actual speed on skin was calculated form the actual distance of the two PFs on skin and the ICMS train duration (Δdistance/Duration). Some results from this task are illustrated in FIG. 22B.

Speed discrimination was also assessed modulating other ICMS train parameters than ICMS train duration or stimulus onset. Indeed, C1 was asked to discriminate speed when Inter-Stimulus-Interval (ISI) or time of overlap (TO) between trains were varied. The former was modulated between Oms and 200 ms and the latter between Oms and 1000 ms.

In each speed discrimination task, multiple electrodes were tested on D2 and D3.

### Letter Forms Encoding and Discrimination Task

To assess the participants' ability to make complex perceptual discriminations between different electrical stimulation sequences a forced choice discrimination task was used. Before discrimination testing, the participants drew the perceived pattern to multichannel stimulation several times. 3 to 6 PFs aligned on D2 in order to create tactile shapes resembling letters (T, L, C, O, I) were selected. 500 ms ICMS trains were delivered simultaneously from all the selected channels or sequentially from each individual electrode depending by the encoding condition. During each trial of the discrimination task, a single sequence was presented and C1 gave a verbal report to indicate which of the letters the subject had perceived. Sequences were presented in pseudo-random order. Data were obtained over 5 experimental blocks, each consisting of 10 presentations of each stimulus. The frequency of the ICMS stimuli was 100 Hz and amplitude was 80 μA. The statistical significance of the accuracy values was obtained using the binom.test( ) function in “R”. To calculated complexity, number of bars were presented considering those necessary to draw the specific letter on the skin. Some results from this task are illustrated in FIGS. 23A and 23B.

### Psychometric Functions

Psychometric functions were fit with a logistic function:

$$p = \frac{1}{1 + {e}^{c * ( x - μ )}}$$

Where p is the probability of judging the comparison stimulus as faster than the standard, x is the train duration, c is the slope, and u the point of subjective equality (PSE). The just noticeable difference (JND) is half the difference between the amplitudes that yield ap of 0.25 and 0.75.

### Cortical Arrangements

To depict the arrangement of the electrodes adopted in each task, this disclosure reported anatomical MRIs with arrays superimposed whose location was based on intra-operative photos (both C1 and C2). The distances on the cortex (inter-electrode distances) and those on skin (inter-centroids PF distances) were calculated and compared. Pearson's Correlation of the perceived and cortical edge length (normalized data) was measured. In the motion encoding, arrows indicate the directions of motion on skin and the sequential order of activation of the adopted electrodes on the arrays (lateral and medial).

### V. Conclusion

It should be understood that arrangements described herein are for purposes of example only. As such, those skilled in the art will appreciate that other arrangements and other elements (e.g., machines, interfaces, operations, orders, and groupings of operations, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and implementations have been disclosed herein, other aspects and implementations will be apparent to those skilled in the art. The various aspects and implementations disclosed herein are for purposes of illustration and are not intended to be limiting, with the true scope being indicated by the following claims, along with the full scope of equivalents to which such claims are entitled. It is also to be understood that the terminology used herein is for the purpose of describing particular implementations only, and is not intended to be limiting.

1. A system, comprising:
an implantable multichannel array that comprises a plurality of channels, each configured to provide stimulation to a respective portion of a somatosensory cortex of a user in which the multichannel array is implanted; and
a controller that is operably coupled to the implantable multichannel array and that comprises one or more processors, wherein the controller is configured to perform controller operations comprising:
obtaining a first time-varying input;
based on the first time-varying input, determining a first time-varying stimulus profile; and
providing, via a first subset of channels of the multichannel array, synchronized stimulation having a magnitude that varies according to the first time-varying stimulus profile, wherein the first subset of channels consists of channels that provide stimulation to respective portions of the somatosensory cortex of the user such that respective perceived locations of the stimulus provided by each channel of the first subset of channels at least partially overlap.

2. The system of claim 1, further comprising a sensor and a prosthetic hand, wherein the time-varying input is a time-varying input force, wherein the controller operations further comprise operating the sensor to detect the time-varying input force, and wherein the prosthetic hand comprises the sensor such that the time-varying input force is a force experienced by a portion of the prosthetic hand.

3-4. (canceled)

5. The system of claim 1, wherein determining the first time-varying stimulation profile based on the first time-varying input comprises determining the first time-varying stimulation profile such that the first time-varying stimulation profile is increased by increases in the magnitude of the first time-varying input and by transient changes in the first time-varying input.

6. The system of claim 5, wherein determining the first time-varying stimulus profile based on the first time-varying input comprises determining a weighted sum of (i) the first time-varying input and (ii) a derivative of the first time-varying input.

7. The system of claim 1, wherein the controller operations further comprise:
obtaining a second time-varying input;
based on the second time-varying input, determining a second time-varying stimulus profile; and
providing, via a second subset of channels of the multichannel array, synchronized stimulation having a magnitude that varies according to the second time-varying stimulus profile, wherein the second subset of channels consists of channels that provide stimulation to respective portions of the somatosensory cortex of the user such that respective perceived locations of the stimulus provided by each channel of the second subset at least partially overlap.

8. The system of claim 7, wherein the system further comprises a memory that contains a record of (i) a record of the identity of the channels of the first subset and (ii) a record of the identity of the channels of the second subset, wherein providing synchronized stimulation having a magnitude that varies according to the first time-varying stimulus profile via the first subset of channels comprises accessing the record of the identity of the channels of the first subset and responsively providing the synchronized stimulation thereto, and wherein providing synchronized stimulation having a magnitude that varies according to the second time-varying stimulus profile via the second subset of channels comprises accessing the record of the identity of the channels of the second subset and responsively providing the synchronized stimulation thereto.

9. The system of claim 1, wherein the first subset of channels comprises at least four channels of the multichannel array.

10. The system of claim 1, wherein the system is operable to provide graded stimulation, via the first subset of channels, in a manner that permits the user in which the multichannel array is implanted to perceive a responsive stimulus in a manner that permits the user to distinguish at least 20 different levels of the stimulus.

11. The system of claim 1, wherein the controller operations further comprise:
obtaining a direction and a speed of a motion;
based on the direction and speed of motion, determining a third time-varying stimulus profile for a first channel of the multichannel array and a fourth time-varying stimulus profile for a second channel of the multichannel array, wherein the third time-varying stimulus profile includes an amount of stimulus prior to stimulus of the fourth time-varying stimulus profile, and wherein identities of the first channel and second channel are selected based on the direction of the motion; and
providing, via the first channel, stimulation having a magnitude that varies according to the third time-varying stimulus profile; and
providing, via the second channel, stimulation having a magnitude that varies according to the fourth time-varying stimulus profile.

12. The system of claim 11, wherein the controller determining the third time-varying stimulus profile for the first channel and the fourth time-varying stimulus profile for the second channel comprises the controller selecting the first channel and the second channel such that a distance between respective perceived locations of the stimulus provided by the first channel and the second channel corresponds to a magnitude of the speed of the motion.

13. The system of claim 11, wherein the controller determining the third time-varying stimulus profile and the fourth time-varying stimulus profile comprises determining a duration of stimulus in the third time-varying stimulus profile relative to a duration of stimulus in the fourth time-varying stimulus profile such that lower speeds result longer durations of the stimulus in the third time-varying stimulus profile.

14. The system of claim 11, wherein stimulus in the third time-varying stimulus profile overlaps with stimulus in the fourth time-varying stimulus profile by one or fewer pulses of stimulation, and wherein an inter-stimulus interval between stimulus in the third time-varying stimulus profile and stimulus in the fourth time-varying stimulus profile is less than 500 milliseconds.

15. (canceled)

16. The system of claim 1, wherein the controller operations further comprise:
obtaining an orientation of an edge;
based on the edge, determining a fifth stimulus profile; and
providing, via a third subset of channels of the multichannel array, synchronized stimulation having a magnitude that corresponds to the fifth stimulus profile, wherein the third subset of channels consists of channels that provide stimulation to respective portions of the somatosensory cortex of the user such that respective perceived locations of the stimulus provided by each channel of the third subset of channels are arranged along a direction based on the orientation of the edge.

17-32. (canceled)

33. A method comprising:
obtaining a first time-varying input;
based on the first time-varying input, determining a first time-varying stimulus profile; and
providing, via a first subset of channels of an implantable multichannel array implanted in a somatosensory cortex of a user, synchronized stimulation having a magnitude that varies according to the first time-varying stimulus profile, wherein the first subset of channels consists of channels that provide stimulation to respective portions of the somatosensory cortex of the user such that respective perceived locations of the stimulus provided by each channel of the first subset of channels at least partially overlap.

34. The method of claim 33, wherein the time-varying input is a time-varying input force, wherein the method further comprises operating a sensor to detect the time-varying input force, and wherein the sensor is part of a prosthetic hand such that the time-varying input force is a force experienced by a portion of the prosthetic hand.

35-37. (canceled)

38. The method of claim 33, wherein determining the first time-varying stimulus profile based on the first time-varying input comprises determining a weighted sum of (i) the first time-varying input and (ii) a derivative of the first time-varying input.

39. The method of claim 33, further comprising:
obtaining a second time-varying input;
based on the second time-varying input, determining a second time-varying stimulus profile; and
providing, via a second subset of channels of the multichannel array, synchronized stimulation having a magnitude that varies according to the second time-varying stimulus profile, wherein the second subset of channels consists of channels that provide stimulation to respective portions of the somatosensory cortex of the user such that respective perceived locations of the stimulus provided by each channel of the second subset at least partially overlap.

40. (canceled)

41. The method of claim 33, wherein the first subset of channels comprises at least four channels of the multichannel array.

42. (canceled)

43. The method of claim 33, further comprising:
providing stimulation via each of the channels of the multichannel array during respective different, non-overlapping periods of time;
based on perceptions experienced by the user during the different, non-overlapping periods of time, determining respective locations of the perceptions experienced by the user, relative to a body part of the user, in response to stimulation by respective channels of the multichannel array; and
based on the locations of the perceptions experienced by the user, determining the first subset of channels.

44-60. (canceled)

60. A non-transitory computer readable medium having stored thereon program instructions executable by at least one processor to cause the at least one processor to perform operations comprising:
obtaining a first time-varying input;
based on the first time-varying input, determining a first time-varying stimulus profile; and
providing, via a first subset of channels of an implantable multichannel array implanted in a somatosensory cortex of a user, synchronized stimulation having a magnitude that varies according to the first time-varying stimulus profile, wherein the first subset of channels consists of channels that provide stimulation to respective portions of the somatosensory cortex of the user such that respective perceived locations of the stimulus provided by each channel of the first subset of channels at least partially overlap.
